(19) United States
(12) Patent Application Publication
(10) Pub. No.: US 2026/0312609 A1
(43) Pub. Date: Oct. 8, 2026

(54) DETECTING AND TRANSMITTING EVENTS ASSOCIATED WITH A SUBJECT

(71) Applicant: Panasonic Well LLC, Palo Alto, CA (US)

(72) Inventor: Ramin Firoozye, San Francisco, CA (US)

(21) Appl. No.: 19/276,667

(22) Filed: Jul. 22, 2025

Related U.S. Application Data
(60) Provisional application No. 63/783,798, filed on Apr. 4, 2025.

(51) Int. Cl. H04B 1/10; G06F 3/0484

(52) U.S. Cl. CPC H04B 1/1027; G06F 3/0484

(57) ABSTRACT

Systems and methods for throttling transmitting events associated with a subject are provided. A computer-implemented method can include receiving sensor data collected by one or more sensors associated with a sender device. The computer-implemented method can also include determining an occurrence of an event associated with a subject of the sender device. In some instances, the event is determined based on the sensor data, and the event is associated with a first event type. The computer-implemented method can also include identifying an event-suppression configuration associated with a receiver device, in which the event-suppression configuration identifies a set of event types. The computer-implemented method can also include applying the event-suppression configuration to the event associated with the subject. The computer-implemented method can also include determining that the set of event types includes the first event type.

### CROSS-REFERENCES TO RELATED APPLICATIONS

The present patent application claims the priority benefit of U.S. provisional patent application No. 63/783,798 filed Apr. 4, 2025 and is related to U.S. Patent Applications Ser. No. 19/276,082 filed Jul. 22, 2025 entitled “SYSTEMS AND METHODS FOR AUTOMATED REVERSE EVENT DETECTION”, Ser. No. 19/276,143 filed Jul. 22, 2025 entitled “CONTINUOUS SENSOR MONITORING FOR IDENTIFICATION OF PREDICTIVE ANOMALIES”, Ser. No. 19/276,187 filed Jul. 22, 2025 entitled “AUTOMATED DISAMBIGUATION OF USERS IN A MULTI-TENANT ENVIRONMENT BASED ON SENSOR DATA”, U.S. Provisional Application 63/783,806 filed Apr. 4, 2025 entitled “SYSTEMS AND METHODS FOR AUTOMATED REVERSE EVENT DETECTION”, U.S. Provisional Application 63/783,800 filed Apr. 4, 2025 entitled “CONTINUOUS SENSOR MONITORING FOR IDENTIFICATION OF PREDICTIVE ANOMALIES” and U.S. Provisional Application 63/783,809 filed Apr. 4, 2025 entitled “AUTOMATED DISAMBIGUATION OF USERS IN A MULTI-TENANT ENVIRONMENT BASED ON SENSOR DATA”, the disclosures of which are incorporated herein by reference in its entirety for all purposes.

### FIELD

The present disclosure relates generally to processing of events detected by sensor devices. In one example, the systems and methods described herein may be used to suppress transmission or presentation of events on one or more receiver devices based on event-suppression configurations.

### SUMMARY

Disclosed embodiments may provide techniques for throttling transmitting events associated with a subject. A computer-implemented method can include receiving sensor data collected by one or more sensors associated with a sender device. The computer-implemented method can also include determining an occurrence of an event associated with a subject of the sender device. In some instances, the event is determined based on the sensor data, and the event is associated with a first event type. The computer-implemented method can also include identifying an event-suppression configuration associated with a receiver device, in which the event-suppression configuration identifies a set of event types. The computer-implemented method can also include applying the event-suppression configuration to the event associated with the subject. In some instances, applying the event-suppression configuration includes determining whether the set of event types includes the first event type. The computer-implemented method can also include determining that the set of event types includes the first event type. The computer-implemented method can also include suppressing transmission of the event to the receiver device.

In an embodiment, a system comprises one or more processors and memory including instructions that, as a result of being executed by the one or more processors, cause the system to perform the processes described herein. In another embodiment, a non-transitory computer-readable storage medium stores thereon executable instructions that, as a result of being executed by one or more processors of a computer system, cause the computer system to perform the processes described herein.

Various embodiments of the disclosure are discussed in detail below. While specific implementations are discussed, it should be understood that this is done for illustration purposes only. A person skilled in the relevant art will recognize that other components and configurations can be used without parting from the spirit and scope of the disclosure. Thus, the following description and drawings are illustrative and are not to be construed as limiting. Numerous specific details are described to provide a thorough understanding of the disclosure. However, in certain instances, well-known or conventional details are not described in order to avoid obscuring the description. References to one or an embodiment in the present disclosure can be references to the same embodiment or any embodiment; and, such references mean at least one of the embodiments.

Reference to “one embodiment” or “an embodiment” means that a particular feature, structure, or characteristic described in connection with the embodiment is included in at least one embodiment of the disclosure. The appearances of the phrase “in one embodiment” in various places in the specification are not necessarily all referring to the same embodiment, nor are separate or alternative embodiments mutually exclusive of other embodiments. Moreover, various features are described which can be exhibited by some embodiments and not by others.

The terms used in this specification generally have their ordinary meanings in the art, within the context of the disclosure, and in the specific context where each term is used. Alternative language and synonyms can be used for any one or more of the terms discussed herein, and no special significance should be placed upon whether or not a term is elaborated or discussed herein. In some cases, synonyms for certain terms are provided. A recital of one or more synonyms does not exclude the use of other synonyms. The use of examples anywhere in this specification including examples of any terms discussed herein is illustrative only, and is not intended to further limit the scope and meaning of the disclosure or of any example term. Likewise, the disclosure is not limited to various embodiments given in this specification.

Without intent to limit the scope of the disclosure, examples of instruments, apparatus, methods and their related results according to the embodiments of the present disclosure are given below. Note that titles or subtitles can be used in the examples for convenience of a reader, which in no way should limit the scope of the disclosure. Unless otherwise defined, technical and scientific terms used herein have the meaning as commonly understood by one of ordinary skill in the art to which this disclosure pertains. In the case of conflict, the present document, including definitions will control.

Additional features and advantages of the disclosure will be set forth in the description which follows, and in part will be obvious from the description, or can be learned by practice of the herein disclosed principles. The features and advantages of the disclosure can be realized and obtained by means of the instruments and combinations particularly pointed out in the appended claims. These and other features of the disclosure will become more fully apparent from the following description and appended claims, or can be learned by the practice of the principles set forth herein.

### BRIEF DESCRIPTION OF THE DRAWINGS

Illustrative embodiments are described in detail below with reference to the following figures.

FIG. 1 illustrates a schematic diagram for generating and transmitting events associated with a subject, according to some embodiments.

FIG. 2 illustrates an example schematic diagram for generating events based on various types of input data, according to some embodiments.

FIG. 3 shows an example screenshot of a user interface for presenting events transmitted by a sender device, according to some embodiments.

FIG. 4 illustrates an example schematic diagram for throttling events by a sender device, according to some embodiments.

FIG. 5 shows an illustrative example of a process for throttling events by a sender device, in accordance with some embodiments.

FIG. 6 illustrates an example schematic diagram for throttling events by a receiver device, according to some embodiments.

FIG. 8 shows a computing system architecture including various components in electrical communication with each other using a connection in accordance with various embodiments.

In the appended figures, similar components and/or features can have the same reference label. Further, various components of the same type can be distinguished by following the reference label by a dash and a second label that distinguishes among the similar components. If only the first reference label is used in the specification, the description is applicable to any one of the similar components having the same first reference label irrespective of the second reference label.

### DETAILED DESCRIPTION

In the following description, for the purposes of explanation, specific details are set forth in order to provide a thorough understanding of certain inventive embodiments. However, it will be apparent that various embodiments may be practiced without these specific details. The figures and description are not intended to be restrictive. The word “exemplary” is used herein to mean “serving as an example, instance, or illustration.” Any embodiment or design described herein as “exemplary” is not necessarily to be construed as preferred or advantageous over other embodiments or designs.

Software applications implemented for tracking day-to-day user activity and events play a critical role in monitoring and enhancing individual well-being. These applications meticulously log various activities and incidents, providing a comprehensive overview of a subject's daily routine and any deviations from their normal behavior. This continuous stream of data allows caregivers to gain valuable insights into the subject's health status in real-time. By analyzing the tracked data, caregivers can identify patterns, detect early signs of potential health issues, and make informed decisions to ensure the subject's well-being. This proactive approach not only enhances the quality of care but also promotes a sense of security for both the subject and their caregivers.

The range of events tracked by these applications can vary significantly, from minor occurrences to severe medical emergencies. For instance, a minor event may be a slight deviation in daily physical activity, while a severe event could be a fall or a sudden health deterioration. In case of severe events, some existing systems are often equipped with alerting systems that immediately notify caregivers via their devices. These instant notifications can enable caregivers to respond promptly and provide necessary medical attention, potentially averting critical situations. This alert mechanism is particularly crucial for vulnerable populations, such as the elderly or individuals with chronic health conditions, as it ensures timely intervention and enhances their overall safety and well-being. However, such alerting systems are incapable of managing an overload of notifications or sequence of events (especially from different sources) that can overwhelm caregivers associated with receiver devices or the monitoring system.

To address the above challenges, the present techniques can be configured to limit and filter event transmission by different devices. In particular, the present techniques can include event throttling that can manage the flow of health data of subjects, thus preventing an overload of data that can overwhelm caregivers associated with receiver devices or the monitoring system. The present techniques can allow the sender device to adjust the amount of event information being transmitted to the receiver devices, at which the receiver devices can also set thresholds and filters based on their specific needs and preferences. For example, a caregiver associated with the receiver device may request to only transmit events related to significant deviations in vital signs, while filtering out less critical events (e.g., missing a prescription medication for a day). Event throttling can thus ensure that the most relevant and important information is communicated, thereby enabling caregivers to focus on what matters without being distracted by excessive or redundant notifications.

The present techniques can also include event cascading to further manage flow of notifications to the receiver devices. Event cascading can include a process by which the events can be transmitted in a cascading sequence from one receiver device to another receiver device based on user-customized settings or the absence of actions. In a cascading configuration, a given event may initially be sent to a first receiver device. If an acknowledgment or action associated with the event is not performed by the first receiver device within a predefined timeframe (e.g., five minutes), the event can then be routed to a second receiver device. The above process can be iterated through different receiver devices that are configured to receive the events from the sender device. The cascading process can ensure that if one caregiver is unavailable or unable to respond, another caregiver will be notified to maintain a continuous chain of communication and response. Event cascading can be particularly useful in situations where immediate attention is required, ensuring that no event goes unnoticed and that the subject receives timely assistance. In some instances, all receiver devices can be notified simultaneously based on the severity of the event. In such scenarios, the present techniques can facilitate communication channels in which the multiple caregivers can coordinate and collaborate in responding to the event.

The present techniques offer significant improvements over existing methods by optimizing the transmission of notifications in a way that enhances both efficiency and effectiveness. By reducing the overall number of notifications being transmitted, caregivers are less likely to become overwhelmed by excessive alerts, allowing them to focus on critical situations that require immediate attention. This reduction in notifications also leads to a more efficient use of computing and networking resources across both sender and receiver devices, as fewer messages mean lower data transmission, reduced processing overhead, and decreased battery consumption, particularly in mobile or resource-constrained environments. Additionally, by prioritizing the transmission of notifications related to relevant and severe events, the present techniques ensure that first responders receive timely and actionable information, improving their ability to intervene effectively in emergency situations. This targeted approach not only enhances response times but also reduces alert fatigue, ensuring that urgent notifications are recognized and acted upon with higher accuracy.

### I. Detecting and Transmitting Events Associated with a Subject

### A. Detecting and Presenting Events

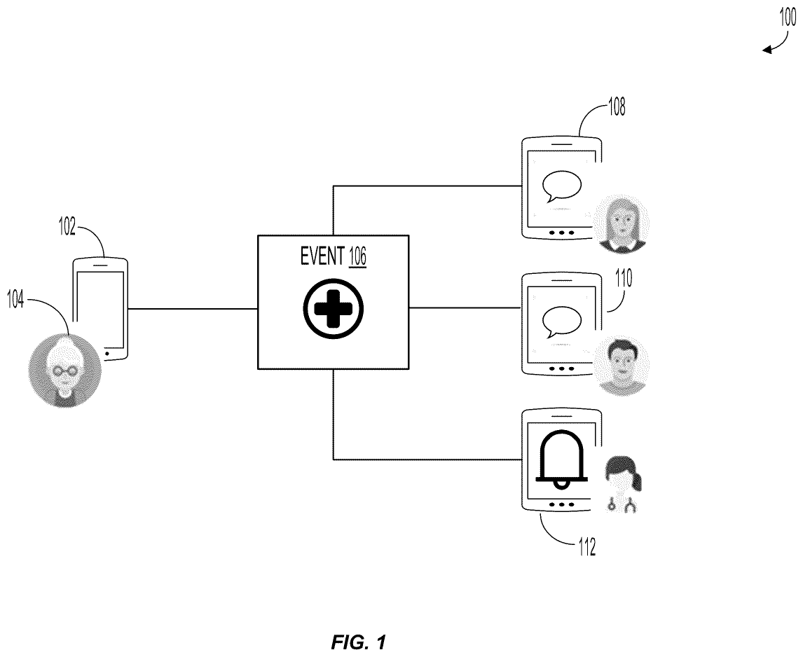

FIG. 1 illustrates a schematic diagram 100 for generating and transmitting events associated with a subject, according to some embodiments. As shown in FIG. 1, a sender device 102 associated with a subject 104 can transmit an event 106 to receiver devices 108-112. The sender device 102 can be worn or used by the subject 104 can be configured to include one or more sensors that continuously and automatically capture various events. In some instances, the sender device 102 can be a sensor device configured to collect sensor data from a surrounding environment, detect the event 106, and send the event 106 to the receiver devices 108-112.

In some instances, the sender device 102 can correspond to a single node. Additionally or alternatively, the sender device can be a type of intermediary always-on device configured to aggregate a number of events to identify common patterns (hereinafter referred to as a “gateway” device). The gateway device can be configured to collect sensor data from environment sensors, including temperature, humidity, air quality, and motion (for example). The gateway device can process the sensor data in real time using an embedded processor with edge computing capabilities to identify detect events, such as sudden temperature spikes, hazardous gas leaks, or absence of subject movement (which potentially indicate an adverse event).

The event 106 can identify various aspects of a subject's daily activities and health metrics, providing relevant data for caregivers and health-monitoring systems. For example, the event 106 can include vital signs, types of activities, activity levels, or specific alerts such as medication reminders or emergency signals. The event 106 can identify a wide range of activities and incidents, from routine health check events such as heart rate monitoring to critical alerts such as falls or emergency button activations. Other activities or routines identified by the event 106 can include but are not limited to daily activities such as eating, showering, and going for a walk, etc., which may also be indicators of whether someone's patterns of activity have worsened.

In some instances, the event 106 can be a raw event or a synthetic event. The raw event can be an initial, unprocessed data measured or recorded for the subject 104. The raw event can include a wide range of data points, such as clinical measurements performed by healthcare providers, diagnostic results from lab tests, imaging studies, administered treatments, types of surgeries performed, adverse events (e.g., side effects). The raw event can also include ambient environmental data as well as from sensors worn on body, embedded, or smart-home nodes. In some instances, the raw event includes events reported by the subject 104, in which the subject 104 describes his or her symptoms, quality of life, or other health-related issues. In addition, the raw event can include physiological parameters (e.g., weight, blood pressure) of the subject 104 using one or more medical devices, including sensors. Raw events can be collected in real-time and may quickly provide meaningful information regarding the subject 104. On the other hand, the raw events can include errors, inconsistencies, or redundancies that may need to be further processed before the raw event can be used for analysis or reporting purposes.

The synthetic event can be generated based on aggregation and interpretation of one or more raw events. Unlike raw events, which are determined based on direct measurements and/or ambient data from sensors (e.g., motion sensors, heart rate monitors), synthetic events can be determined based on analyzing the raw data to create more meaningful and actionable information. For example, if a given raw event describes movement of the subject 104 or identifies ambient data associated with sound and mmWave motion data, the synthetic event can indicate a medically-adverse event due to the subject 104 falling to the ground. In another example, if the raw event detects a change in gait or an “absence” of movement, the synthetic event can indicate a current or future medically-adverse event. Additionally or alternatively, other variations in data including variation in “rate of change” of data (e.g., second-order derivatives) can be monitored to generate the synthetic event. Various types of values using filtering/pattern-matching algorithms can be used to detect synthetic events.

In some instances, a data-aggregation server receives the raw events measured from various sensors, aggregates the raw events, and generates a corresponding synthetic event. The synthetic event can be interpreted by the data-aggregation server (or another server) to determine whether the synthetic event should be transmitted to the receiver devices 108-112. The conversion of raw data into synthetic events can facilitate more precise health monitoring of the subject 104. Additionally or alternatively, complex synthetic events can be generated by integrating data from multiple sources, offering a comprehensive view of the activities and health status of the subject 104. For example, combining motion-sensor data with heart-rate patterns can identify sleep stages or detect unusual activity levels of the subject 104.

In some instances, the event 106 can be associated with an event classification. The event classification can identify one or more characteristics associated with the event 106. The event classification can identify a type for the event 106, such as health metrics, activity levels, and medication reminders. The event classification can facilitate caregivers to quickly assess the situation and take appropriate actions for the subject 104.

In some instances, a severity metric is generated for the corresponding event 106. If the severity metric exceeds a particular threshold, the sender device 102 can transmit the event 106 to one or more of the receiver devices 108-112. The real-time transmission of the event 106 can facilitate caregivers to respond quickly and appropriately to the subject's situation, which can reduce the risk of medically adverse events for the subject 104. The real-time data transmission of the event 106 can further reduce the burden of the subject 104, allowing the subject 104 to go about his or her daily activities while still being closely monitored by healthcare facilities. Additionally or alternatively, if the severity metric does not exceed the threshold, the sender device 102 can suppress the event 106 from being transmitted to the receiver devices 108-112 to reduce network traffic and avoid excess events to be transmitted to the corresponding caregivers. In effect, the caregivers can be prepared to respond only to the events that can cause medically adverse events.

As an illustrative example, the sender device 102 can be configured to continuously monitor heart rate of the subject 104. The events can be suppressed for the heart rates that fall within a normal range. If the heart rate deviates significantly from the norm or shows irregular movement patterns, the sender device 102 can generate and transmit in real-time the event 106 to the receiver devices 108-112. Additionally or alternatively, the sender device 102 generates the event 106 to be presented on an interface of the sender device 102 and any recommended actions to be performed in response to the event 106. For example, the sender device 102 can be configured to display reminders for the subject 104 to take his or her medication at specific times in response to detection of the event 106. If the scheduled medication is missed, additional events associated with higher severity metrics can be generated and transmitted.

The receiver devices 108-112 can be configured to receive the event 106 generated by the sender device 102. In some instances, the receiver devices 108-112 can be associated with one or more of caregivers or healthcare providers. The caregivers can access and review the event 106 through various interfaces of the receiver devices 108-112, including mobile devices, tablets, or dedicated medical monitoring systems. The receiver devices 108-112 can facilitate the caregivers and healthcare providers to stay updated on the subject's condition regardless of their locations. In some instances, the events associated with high severity metrics (e.g., severity metrics that exceed the threshold) can trigger one or more alerts to be generated by the receiver devices 108-112. The alerts can be issued to notify the caregivers and/or healthcare providers of any significant changes or emergencies. In some instances, at least one of the receiver devices 108-112 can be configured as a centralized hub device, to which the data associated with the event 106 can be analyzed.

The event 106 can be transmitted to the receiver devices 108-112 using two different implementations: (i) sending events directly to another server (e.g., a cloud server) in real-time; and (ii) gathering and analyzing the events using a local device (e.g., the sender device 102, a gateway device within the subject's environment) before relaying the events to the receiver devices 108-112. In the real-time server transmission approach, subject data collected using various data sources (e.g., sensor data, manual input) can be immediately transmitted to the cloud-based, server system. For example, sensor data associated with the subject 104 can be packaged with relevant metadata, including timestamps and source identifiers. The packaged data can then be transmitted over a communication network to the server system. The server system can be configured to handle large volumes of data, providing robust storage and processing capabilities. Once the subject data reaches the server system, the server system can analyze and transform the subject data into the event 106, which can then be availed to the receiver devices 108-112 in real-time. In some instances, the server system determines whether to suppress the event 106 from transmission (e.g., low severity metric) or transmit the event 106 to the receiver devices 108-112 (e.g., high severity metric). The server-system approach can minimize processing time for generating the event 106 and ensure that the latest information associated with the subject 104 is always available, allowing for timely interventions and continuous monitoring. The real-time aspect is particularly beneficial for events that require immediate attention, as it minimizes the delay between event generation and caregiver notification.

Additionally or alternatively, the subject data can be accessed by a local device (e.g., the sender device 102, a gateway device), which can be processed to generate the event 106. The local device can then relay the event 106 directly to the receiver devices 108-112. In some instances, local devices for generating the event 106 can include peripheral devices, smart speakers, or gateway devices associated with the sender device 102, in which the local devices can aggregate the data collected by various sensors within the subject's environment and generate the event 106. These local devices can perform initial data processing tasks, such as filtering noise, detecting anomalies, and synthesizing raw data into higher-level events. The local processing can reduce the amount of data that needs to be transmitted to the receiver devices 108-112, as only significant events can be transmitted out. The local-processing configuration can thus optimize bandwidth usage and enhance data security. In addition, the local-processing configuration can be advantageous in scenarios in which internet connectivity is intermittent, or where data privacy is a major concern, as the local-processing configuration allows for more control over the data flow.

In some instances, both configurations can be implemented together. For example, a hybrid configuration can use local processing for routine data and real-time cloud transmission for critical events. By implementing various transmission configurations, the healthcare monitoring system can ensure reliable, timely, and secure delivery of important health events to the caregivers and healthcare providers.

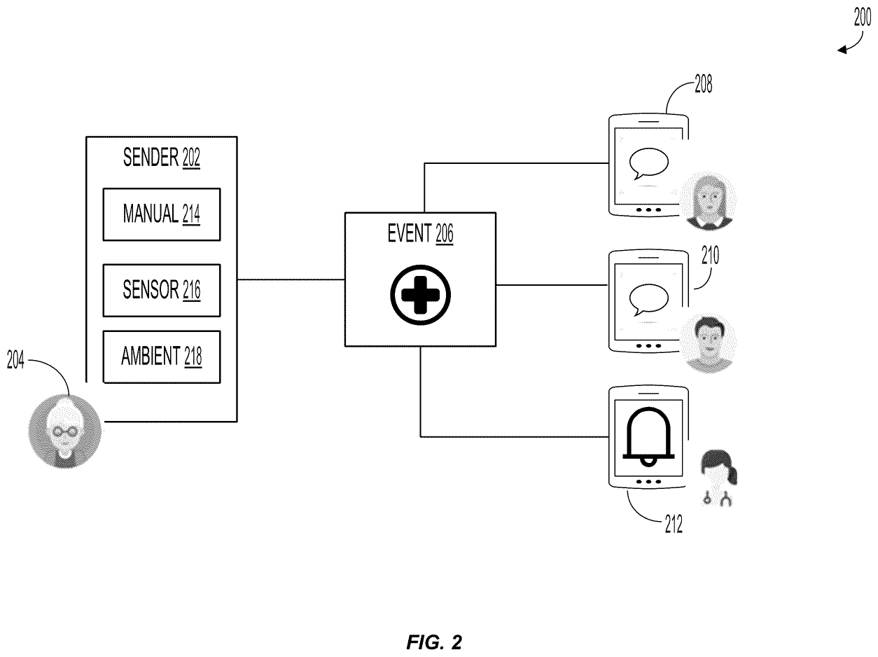

FIG. 2 illustrates an example schematic diagram 200 for generating events based on various types of input data, according to some embodiments. As shown in FIG. 2, the sender device 202 can process various types of input data associated with a subject 204 to generate a corresponding event 206. The sender device 202 can then transmit the event 206 to one or more of receiver devices 208-212. For example, the input data for generating the event 206 can include self-reported data 214, sensor data 216, and ambient data 218. Regarding the self-reported data 214, a user interface of the sender device 202 can be configured to enable the subject 204 to manually input his or her health data, including symptoms, medication intake, or any other significant health-related information. When the subject 204 submits the self-reported data 214, the sender device 202 can attach the self-reported data 214 with corresponding metadata, such as timestamps when the self-reported data 214 was generated and user identifiers associated with the subject 204. The sender device 202 can then locally generate the event 206 based on the self-reported data 214 and the metadata. Additionally or alternatively, the sender device 202 can transmit the self-reported data 214 and the metadata to an event-generation system, at which the event-generation system can generate the event 206 for the subject 204.

The sensor data 216 can identify physiological characteristics associated with the subject 204, which can be collected using various sensors associated with the sender device 202. The sensors can include accessory devices (e.g., smartwatch, heart-rate monitor, pedometer) worn by the subject 204. The sensors can continuously collect raw data and transmit the raw data to a local processing device (e.g., the sender device 202) or directly to another server for further processing. In some instances, the sender device 202 filters out noise, detects anomalies, and processes the raw data into meaningful data for identifying various information associated with the event 206. As an illustrative example, the sensor data 216 can include sensor data collected from a heart-rate monitor that is configured to track heart rate, blood pressure, or motion of the subject 204. The heart-rate monitor can transmit the heart-rate data to the sender device 202, at which the sender device 202 can process the data to identify any irregular heartbeats and transmits the event 206 to receiver devices 208-212 if anomalies are detected. The receiver devices 208-212 can then issue an alert associated with the event 206, such that corresponding caregivers can perform any appropriate action in response to the anomalies.

Ambient data 218 can include data collected from devices located at the environment associated with the subject 204. The ambient data 218 can be collected from sensors such as motion detectors, sound sensors, or smart home devices. The sensors can operate passively, collecting the ambient data 218 without requiring user interaction. The collected ambient data 218 can be transmitted to the sensor device 202 or the sender device (e.g., a gateway device, another server), at which the ambient data 218 can be synthesized to detect specific events (e.g., the event 206). For example, a combination of motion data and sound patterns can help determine if the subject 204 is sleeping, walking, or in distress. The sender device 202 may analyze motion data indicating no movement along with sound sensor data detecting snoring sounds to infer that the subject 204 is sleeping. In another example, a detected rapid motion combined with the sound of footsteps may indicate the subject 204 may be walking in distress.

By integrating the self-reported data 214, the sensor data 216, and the ambient data 218, the sender device 202 can detect events based on comprehensive information about the subject 204. Such integration can ensure that receiver devices 208-212 receive accurate and timely information, enabling the caregivers to provide better care and support for the subjects being monitored. The ability to process and synthesize data from various sources into actionable events can significantly enhance the quality of healthcare monitoring and intervention.

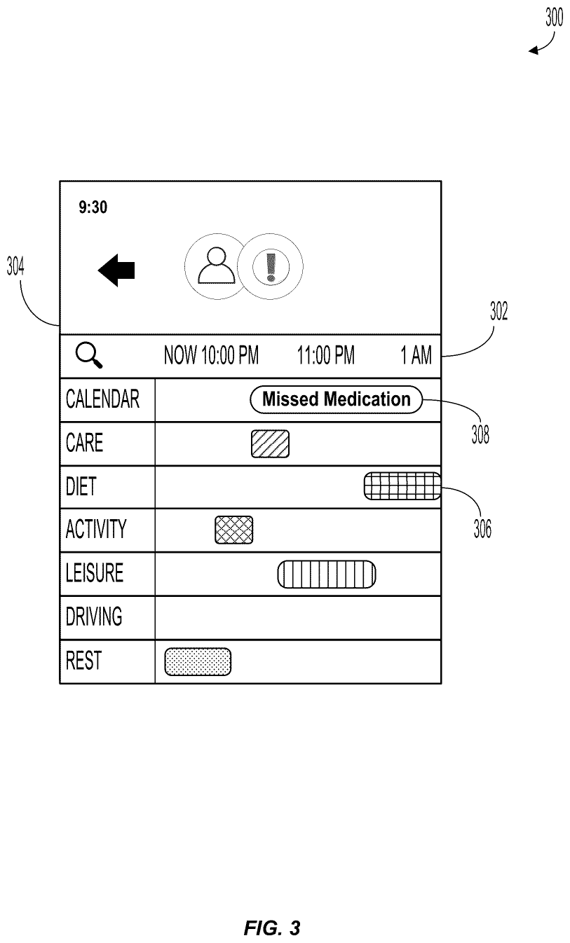

FIG. 3 shows an example screenshot of a user interface 300 for presenting events transmitted by a sender device, according to some embodiments. The user interface 300 shows a timeline 302 that can be used to track occurrence and duration of activities across different time points. The activities can include activities performed or scheduled to be performed by a subject. The timeline 302 includes one or more time points for performing such set of activities. For example, the timeline 302 can include a “Now” time point that indicates the current time, a 10 AM time point, and a 2 PM time point.

The user interface 300 also shows a category element 304 that indicates a corresponding category associated with an activity associated with a subject. The category element 304 can identify a type of activity performed by the subject within one or more time windows of the timeline 302. Additionally or alternatively, the category element 304 can also identify other aspects about the subject, including but not limited to location, parts of body, body systems (i.e. heart vs. eyes), and functionally (i.e. sleeping vs eating vs exercising). The user interface 300 can additionally show an activity duration bar 306 that identifies a duration of activity corresponding to the category element 304. As an illustrative example, the category element 304 corresponding to “care” category includes an activity duration bar identifying that the subject has taken one or more prescription drugs at 10 AM. In another example, the category element 304 corresponding to “leisure” category includes another activity duration bar identifying that the subject has engaged in a leisure activity between 10 AM and 2 PM. Other examples of the category element 304 can include a calendar category, a care category, a diet category, a physical activity category, a leisure category, a drive category, and a rest category.

In some instances, the category element 304 can additionally identify that a particular type of activity was not performed by the subject, in which the particular type of activity was initially scheduled on corresponding time windows of the timeline 302. The indication of the missed activity can be analyzed to determine whether events should be generated then transmitted to one or more corresponding caregivers. If so, the event duration bar 306 can be additionally configured to indicate an event 308. The event 308 can indicate an activity that was performed outside of the scheduled activities of the subject (e.g., a movement pattern during sleeping hours). In some instances, the event 308 can also indicate that the subject has failed to perform a particular scheduled activity (e.g., did not take medication). In such instances, the caregivers or health providers can determine one or more actions in response to reviewing the event 308 presented on the user interface 300. The one or more actions can include contacting subject or initiating a medical emergency call to paramedics.

As an illustrative example, the category element 304 corresponding to “calendar” category includes an activity bar identifying that the subject has missed taking a medication between 10 AM and 2 PM. Based on the type of medication and severity of side effects, an event-processing application can determine an occurrence of a particular event associated with the subject.

Although the events can be generated and frequently transmitted for the benefit of the health and well-being of the subject, there are some instances in which the transmission of certain events to receiver devices needs to be filtered, particularly in scenarios where privacy of the subjects needs to be protected. The present techniques can thus include a user interface control that enables “event throttling.” The event throttling feature can allow the users to fine-tune the amount and types of events being transmitted to receiver devices. Event throttling can limit and filter events, to the extent that it can reduce data overflow and ensure that only relevant and secure information is sent to the receiver devices.

Sender throttling can be configured to manage and control the flow of events being transmitted by a sender device to receiver devices (e.g., caregiver devices, a healthcare monitoring server system). The sender throttling can allow customization of event transmission based on various parameters, ensuring that data privacy is maintained, costs are managed, and unnecessary notifications are minimized. Implementing sender throttling can include configuring rules and filters that dictate when and how events are transmitted. In some instances, machine-learning techniques can be used to further manage the flow of the events from being transmitted to the receiver devices.

B. Event Throttling from the Sender Device

As described herein, the present techniques can be configured to limit and filter event transmission by one or more sender devices. In particular, the present techniques can include event throttling that can manage the flow of health data of subjects, thus preventing an overload of data that can overwhelm caregivers associated with receiver devices or the monitoring system. The present techniques can allow the sender device to adjust the amount of event information being transmitted to the receiver devices, at which the receiver devices can also set thresholds and filters based on their specific needs and preferences. For example, a caregiver associated with the receiver device may request to only transmit events related to significant deviations in vital signs, while filtering out less critical events (e.g., missing a prescription medication for a day). Event throttling can thus ensure that the most relevant and important information is communicated, thereby enabling caregivers to focus on what matters without being distracted by excessive or redundant notifications.

The present techniques can also include event cascading to manage flow of notifications to the receiver devices. Event cascading can include a process by which the events can be transmitted in a cascading sequence from one receiver device to another receiver device based on user-customized settings or the absence of actions. In a cascading configuration, a given event may initially be sent to a first receiver device. If an acknowledgment or action associated with the event is not performed by the first receiver device within a predefined timeframe (e.g., five minutes), the event can then be routed to a second receiver device. The above process can be iterated through different receiver devices that are configured to receive the events from the sender device. The cascading process can ensure that if one caregiver is unavailable or unable to respond, another caregiver will be notified to maintain a continuous chain of communication and response. Event cascading can be particularly useful in situations where immediate attention is required, ensuring that no event goes unnoticed and that the subject receives timely assistance. In some instances, all receiver devices can be notified simultaneously based on the severity of the event. In such scenarios, the present techniques can facilitate communication channels in which the multiple caregivers can coordinate and collaborate in responding to the event.

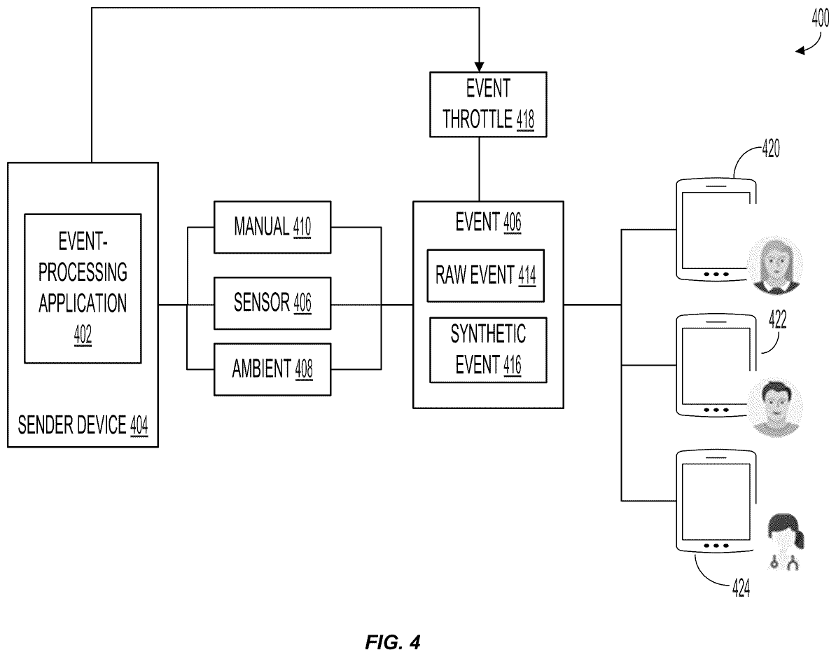

FIG. 4 illustrates an example schematic diagram 400 for throttling events by a sender device, according to some embodiments. In FIG. 4, an event-processing application 402 associated with a sender device 404 can receive sensor data 406 collected by one or more sensors. The one or more sensors can include accessory devices (e.g., smartwatch, heart-rate monitor, pedometer) worn by a subject associated with the sender device 404. The one or more sensors can continuously collect raw data and transmit the raw data to the sender device 404 or directly to another server for further processing. In some instances, the sender device 404 filters out noise, detects anomalies, and processes the sensor data 406 into meaningful data. As an illustrative example, the sensor data 406 can be collected from a heart-rate monitor that is configured to track heart rate, blood pressure, or motion of the subject.

In some instances, the one or more sensors can be associated with an Internet of Things (IoT) sensor network. For example, the event-processing application 402 can communicate with an Internet of Things (IoT) sensor network to receive the sensor data 406 associated with the subject. The IoT sensor network can includes a sensor data layer that includes various IoT sensors that can measure physical surroundings and capture real-time environment changes. The IoT sensors can include temperature, pressure, humidity, level, accelerometer, gas, gyroscopes, motion sensors image, optical sensors, Radiofrequency Identifier (RFID) sensors, and Infra-Red (IR) sensors. The IoT sensors are mainly associated with the microprocessing unit, storage unit, control unit, power system and wireless communication interfaces. The IoT sensor devices are constrained in size, computing power, memory, networking capability and storage space. Wireless communication protocols, such as Wi-Fi, Zig Bee, Bluetooth, Near Frequency Communication (NFC), LE, Thread, and Matter, as well as wired protocols like Ethernet, RS232, CANBus, and LTE/4G mobile technologies, are used for IoT sensor device communication within the sensor network.

In some instances, the event-processing application 402 can additionally receive ambient data 408 that can include data collected from devices located at the environment associated with the subject. The ambient data 408 can be collected from sensors such as motion detectors, sound sensors, or smart home devices. The sensors can operate passively, collecting the ambient data 408 without requiring user interaction. The collected ambient data 408 can be transmitted to the sender device 404 or another server, at which the ambient data 408 can be processed and/or synthesized to detect specific events (e.g., the event 206). For example, a combination of motion data and sound patterns can help determine if the subject is sleeping, walking, or in distress. The sender device 404 may analyze motion data indicating no movement along with sound sensor data detecting snoring sounds to infer that the subject is sleeping. In another example, a detected rapid motion combined with the sound of footsteps may indicate the subject may be walking in distress.

In addition, the event-processing application 402 can additionally receive manual or self-reported data 410. A user interface of the sender device 404 can be configured to enable the subject or a proxy (e.g., a caregiver) to manually input his or her health data, including symptoms, medication intake, or any other significant health-related information. When the subject submits the self-reported data 410, the sender device 404 can attach the self-reported data 410 with corresponding metadata, such as timestamps when the self-reported data 410 was generated and user identifiers associated with the subject.

By integrating the self-reported data 410, the sensor data 406, and the ambient data 408, the sender device 404 can detect events based on comprehensive information about the subject. Such integration can ensure that receiver devices receive accurate and timely information, enabling the caregivers to provide better care and support for the subjects being monitored. The ability to process and synthesize data from various sources into actionable events can significantly enhance the quality of healthcare monitoring and intervention.

The event-processing application 402 can then determine an occurrence of an event 412 associated with a subject of the sender device 404. The event 412 can identify various aspects of a subject's daily activities and health metrics, providing relevant data for caregivers and health-monitoring systems. For example, the event 412 can include vital signs, activity levels, or specific alerts such as medication reminders or emergency signals. The event 412 can identify a wide range of activities and incidents, from routine health check events such as heart rate monitoring to critical alerts such as falls or emergency button activations.

The event 412 can be determined by analyzing the sensor data 406. For example, the event-processing application 402 may preprocess the sensor data 406 and the ambient data 408 (e.g., filtering operations) to remove noise, normalize the sensor data 406 and the ambient data 408, and segment the sensor data 406 and the ambient data 408 into smaller windows. The event-processing application 402 can extract one or more features from each of the preprocessed data segments. Feature extraction can include determining, for each of the preprocessed data segments, statistical features, frequency domain features using techniques such as Fourier Transform, and/or time-domain features including peaks and valleys. In some instances, domain-specific features relevant to the preprocessed data segments can also be extracted.

To detect occurrence of the event, the event-processing application 402 can use threshold-based techniques, in which features that trigger predefined thresholds can be determined as qualified events. In some instances, pattern-recognition techniques can also be used to classify sensor-data segments as events or non-events based on the extracted features. In some instances, validation and testing techniques are used to evaluate the detection performance of events, in which the validation techniques can include identifying evaluation metrics such as accuracy, precision, recall, and F1-score.

If the sensors are associated with the IoT sensor network, the IoT sensor network can include a data fusion layer configured to integrate the sensor data 406, the ambient data 408, and the self-reported data 410, which can be forwarded to a data analysis layer to determine the occurrence of the event. In some instances, the data analysis layer implements data processing techniques to determine the occurrence of the event. Examples of the data processing techniques can include data denoising, missing data imputation (e.g., Gaussian model, spatial and temporal correlation, probabilistic matrix factorization), data outlier detection (e.g., principal component analysis), and data fusion (e.g., Bayesian method).

In some instances, the event 412 can be a raw event 414 or a synthetic event 416. As described herein, the raw event 414 can be an initial, unprocessed data measured or recorded for the subject. The raw event 414 can include a wide range of data points, such as clinical measurements performed by healthcare providers, diagnostic results from lab tests, imaging studies, administered treatments, types of surgeries performed, adverse events (e.g., side effects). In some instances, the raw event 414 includes events reported by the subject, in which the subject describes his or her symptoms, quality of life, or other health-related issues. In addition, the raw event 414 can include physiological parameters (e.g., weight, blood pressure) of the subject using one or more medical devices, including sensors. Raw events 414 can be collected in real-time and may quickly provide meaningful information regarding the subject. On the other hand, the raw events 414 can include errors, inconsistencies, or redundancies that may need to be further processed before the raw event 414 can be used for analysis or reporting purposes.

The synthetic event 416 can be generated based on aggregation and interpretation of one or more raw events 414. Unlike raw events 414, which are determined based on direct measurements from sensors (e.g., motion sensors, heart rate monitors), synthetic events 416 can be determined based on analyzing the raw data to create more meaningful and actionable information. For example, if a given raw event 414 describes movement of the subject, the synthetic event 416 can indicate a medically-adverse event due to the subject falling to the ground. In some instances, a data-aggregation server receives the raw events 414 measured from various sensors, aggregates the raw events 414, and generates a corresponding synthetic event 416. The synthetic event 416 can be interpreted by the data-aggregation server (or another server) to determine whether the synthetic event 416 should be transmitted to the receiver devices. The conversion of raw data into synthetic events 416 can facilitate more precise health monitoring of the subject. Additionally or alternatively, complex synthetic events 416 can be generated by integrating data from multiple sources, offering a comprehensive view of the activities and health status of the subject. For example, combining motion-sensor data 406 with heart-rate patterns can identify sleep stages or detect unusual activity levels of the subject.

In some instances, the event 412 be associated with an event classification. The event classification can identify one or more characteristics associated with the event 412. The event classification can identify a type for the event 412, such as health metrics, activity levels, and medication reminders. The event classification can facilitate caregivers to quickly assess the situation and take appropriate actions for the subject.

In some instances, a severity metric is generated for the corresponding event 412. If the severity metric exceeds a particular threshold, the sender device 404 can transmit the event 412 to one or more of the receiver devices. The real-time transmission of the event 412 can facilitate caregivers to respond quickly and appropriately to the subject's situation, which can reduce the risk of medically adverse events for the subject. The real-time data transmission of the event 412 can further reduce the burden of the subject, allowing the subject to go about his or her daily activities while still being closely monitored by healthcare facilities. Additionally or alternatively, if the severity metric does not exceed the threshold, the sender device 404 can suppress the event 412 from being transmitted to the receiver devices to reduce network traffic and avoid excess events to be transmitted to the corresponding caregivers. In effect, the caregivers can be prepared to respond only to the events that can cause medically adverse events.

In some instances, the event-processing application 402 can determine the occurrence of the event 412 by applying an event-detection machine-learning model to the sensor data 406, ambient data 408, and/or self-reported data 410 to determine the occurrence of the event. In some instances, the event-processing application 402 can also process one or more raw events 412 using the event-detection machine-learning model to generate a synthetic event 414. Examples of the event-detection machine-learning model can include algorithms such as k-means clustering algorithms, fuzzy c-means (FCM) algorithms, expectation-maximization (EM) algorithms, hierarchical clustering algorithms, and density-based spatial clustering of applications with noise (DBSCAN) algorithms, in which the algorithms can be trained using unsupervised learning. Other examples of the event-detection machine-learning model 104 can include, but are not limited to, genetic algorithms, backpropagation, reinforcement learning, decision trees, linear classification, artificial neural networks, anomaly detection, and such. In yet other examples, the event-detection machine-learning model may include regression analysis, dimensionality reduction, metalearning, reinforcement learning, deep learning, and other such algorithms and/or methods. Additionally or alternatively, the event-detection machine-learning model can include anomaly detection algorithms, sush as KNN, random or isolation forest, robust covariance, support vector machine (SVM), stochastic gradient descent (SGD), k-means, or long short-term memory (LSTM). These algorithms can be used in detecting device failures if sensors or gateways show signs of failure.

In some instances, the event-detection machine-learning model is a transformer model (e.g., a large-language model (LLM)) obtained from a models database. In some instances, the event-detection machine-learning model is trained using self-supervised learning based on a large corpus of text data, such that the event-detection machine-learning model can generate the synthetic events. In addition to training the model, various prompts can be used for prompt engineering of the event-detection machine-learning model for generating the synthetic events. Examples of the event-detection machine-learning model can include, but are not limited to, BERT model, Claude LLM, Falcon 40B, Ernie, GPT-3, GPT-3.5, GPT 4, Lamda, and Llama.

An illustrative example process of training and deploying the transformer model (e.g., a GPT model) is as follows. For the training dataset (e.g., the previous input data and event 412), the masked self-attention process can begin by transforming each word in a given training text sequence into three vectors: the query (Q), key (K), and value (V) vectors. A Q vector can represent what information the token is querying about other tokens, a K vector can represent the token's context used to establish relationships with other tokens, and a V vector can represent the token's actual content/information. In some instances, the Q, K, and V vectors can be obtained by multiplying the input embeddings by learned weight matrices.

An attention score for a particular word can be calculated by taking the dot product of the Q vector of the word with the K vectors of all words in the sequence, thereby producing a score that reflects the relevance of each word pair. The attention scores can be used as weights, which can be applied to the Q, K, V vectors to generate a weighted contextual representation of the particular word. Stated differently, the attention score can be used as a weight to transform the Q, K, V vectors of a given word to generate a weighted, computed representation that can be used to train the corresponding transformer model.

In some instances, a mask can be applied to the self-attention mechanism such that a contextual representation of a given token is determined without weights associated with future tokens. As a result, an attention score of a particular token can be adjusted to disregard information from tokens that have not been processed yet. The attention scores can then be scaled by the square root of the key dimension to stabilize training and passed through a softmax function to convert the attention scores into probabilities, ensuring they sum to one. The transformation can identify the most relevant words while downplaying less important ones. The resulting attention weights can then be used to compute a weighted sum of the V vectors, thus producing a new contextual representation for each token that incorporates contextual information from the entire sequence.

To enhance the model's ability to capture various types of relationships, self-attention mechanisms can use multiple sets of Q, K, and V matrices, also referred to as multi-head attention. Each set, or head, can learn different aspects of the relationships within the input data. The outputs from these heads can be concatenated and linearly transformed to form the final self-attention output. This multi-head approach allows the transformer models to simultaneously consider different features and interactions, enriching its understanding of the input sequence.

The transformer model can then be trained using autoregressive language modeling to predict a subsequent token of a target sequence based on the contextual representations that represent the preceding tokens. For each position in the sequence, the transformer model accesses a contextual representation of the token, which was generated using masked self-attention mechanism. The transformer model can then output a probability distribution over a vocabulary for the subsequent token, conditioned on the sequence of preceding tokens. The subsequent token can then be compared with a corresponding token of the training data to calculate a loss. The loss measures the discrepancy between the predicted token and the actual token, providing a signal for the model to adjust its parameters. The loss can then be used to adjust parameters of the transformer model, including the parameters of the Q, K, V matrices.

Through iterative training iterations, the transformer model learns to minimize this loss across the entire training dataset. This process ensures that the model generates coherent and contextually appropriate sequences by leveraging the learned representations and adjusting its parameters based on the training data.

In some instances, the event-processing application 402 can construct one or more prompts that can be submitted with the sensor data 406, ambient data 408, and/or self-reported data 410 to enhance and increase the accuracy of the event 412. As used herein, the term “prompt” can refer to an input sequence generated to direct a corresponding machine-learning model's generation process towards producing a target output. In some instances, a filtering prompt includes a sequence of text tokens in a specific format (e.g., text, XML data, JSON data) and language (e.g., English, Korean).

In some instances, the prompts are machine-generated prompts that are generated by one or more computer systems without user intervention. For example, the one or more filtering prompts can be constructed using prompt engineering. Prompt engineering can include techniques for designing and implementing prompts within a machine-learning system to generate target responses or actions. In some instances, prompt engineering leverages a combination of linguistic approaches, machine-learning algorithms, and domain knowledge to formulate prompts that elicit specific outputs from a corresponding machine-learning model. The prompt engineering process typically begins with an analysis of a target or a problem domain, followed by the formulation of prompts tailored to achieve the desired results.

As an illustrative example for optimizing prompts, a prompt P can be defined as a sequence of tokens, tailored to elicit specific responses from a machine-learning model. The model employs an objective function O(P, R) to evaluate the quality of generated responses R given the prompt P. The responses R can be generated based on a machine-learning language model LM processing the prompt P (e.g., the function LM(P)). Different types of objective functions can be selected depending on the task and targeted output. For example, an objective function can correspond to a text summarization technique using ROUGE scores. In another example, the objective function can correspond to a translation quality assessment technique using BLEU scores. In some instances, optimization techniques like gradient descent or evolutionary algorithms are used iteratively refine the prompt P to maximize O(P, R), to facilitate the model to consistently produce accurate, relevant, and contextually appropriate outputs (e.g., the event 412). For example, the optimal prompt P* can be determine based on maximizing the objective function O:

$$P *= arg max O ( P , LM ( P ) ) \qquad Equation ( 1 )$$

Through the iterative refinement process, prompt engineering enhances the corresponding model's performance across various natural language processing tasks, such as generating the event 412 that are contextually relevant to the sensor data 406, ambient data 408, and/or self-reported data 410.

In some instances, prompt engineering includes a selection of input formats and structures. The input-format selection can include determining the syntactic and semantic characteristics of the prompts that will effectively guide the machine-learning model towards the desired outputs. In some instances, linguistics and computational linguistics can be used to select input formats that are semantically meaningful and contextually relevant. The input-format selection can ensure that the prompts effectively communicate the desired tasks or questions to the machine-learning model. The prompt engineering process can also include an optimization of prompt parameters. The optimization can include fine-tuning various parameters such as prompt length, complexity, and specificity to enhance the machine-learning model's performance on targeted tasks. Different prompt formulations and configurations such as grid search or Bayesian optimization can be implemented to optimize the prompt parameters. Additionally or alternatively, techniques such as zero-shot learning or few-shot learning can be implemented to fine-tune the machine-learning models to generalize from limited prompt examples.

The prompt engineering process can be configured based on an underlying machine-learning model architecture and training data. For example, an appropriate pre-trained machine-learning model architecture (e.g., GPT, BERT, or Transformer) that aligns with the task requirements and available computational resources can be identified for a given task. In some instances, the machine-learning model can be fine-tuned on task-specific data to further improve probability of outputting target responses. Various types of training datasets can be used to train and fine-tune the machine-learning model, so as to enable the machine-learning model to understand and generate responses to prompts accurately.

In some instances, an iterative process of designing, testing, and optimizing prompts is implemented based on feedback from initial model outputs. This iterative approach allows for continuous improvement and refinement of the prompt engineering process, ultimately leading to better-performing machine-learning models. Additionally or alternatively, ongoing monitoring and evaluation of model performance can be used to identify any errors or biases introduced by the prompts and prompt engineering process, in which the feedback data can be generated based on the evaluation. The feedback data can be used to further adjust the parameters of the machine-learning models, such that the machine-learning models can be updated to improve accuracy in generating the target responses.

The event-processing application 402 can apply the trained and fine-tuned machine-learning model to the sensor data 406, ambient data 408, and/or self-reported data 410 to generate the event 412. To begin the deployment process, the event-processing application 402 can tokenize the merged data into a sequence of text tokens. For example, the merged data can be tokenized to provide the following sequence: [“You”, “are”, “an”, “assistant”, “tasked”, . . . ]. In some instances, the machine-learning model uses Byte Pair Encoding (BPE) techniques to further split a single token (e.g., “in”, “sufficient”).

The event-processing application 402 can assign each token with a particular index value in the vocabulary (e.g., “assistant”=E[5]). Then, the event-processing application 402 can convert each token into a vector representation (e.g., an embedding) based on a pre-trained embedding matrix. For example, for a vocabulary size V and embedding dimension dᵢ, the embedding matrix E is of size V×d, in which the vector eᵢ can be generated for the text token tᵢ based on using the index value looking at a corresponding row of embedding matrix E.

$$E : {e}_{i} = E [ {t}_{i} ] \qquad Equation ( 2 )$$

The event-processing application 402 can then process the sequence of embeddings (e₁, e₂, e₃, . . . eₙ) that represent the sequence of tokens by adding positional encodings to account for the order of tokens. In some instances, positional encodings are vectors added to each token embedding to inject information about the position of tokens in the sequence. A matrix X can be formed that includes the sequence of position-encoded vectors.

For the matrix X, the event-processing application 402 can then determine a contextual representation for each position-encoded vector of the matrix X. In particular, for each position-encoded vector, the event-processing application 402 can generate a set of Q, K, V vectors for the position-encoded vector. As described herein, a Q vector can represent what information the token is querying about other tokens, a K vector can represent the token's context used to establish relationships with other tokens, and a V vector can represent the token's actual content/information.

In some instances, to enhance the model's ability to capture various types of relationships, the position-encoded vector can be represented by multiple sets of Q, K, and V matrices (i.e., multi-head attention). Each set of Q, K, V vectors, or head, can learn different aspects of the relationships within the input data. The outputs from these heads can be concatenated and linearly transformed to form the final self-attention output. This multi-head approach allows the transformer models to simultaneously consider different features and interactions, enriching its understanding of the input sequence.

An attention score can be calculated for the set of Q, K, V vectors as follows:

$$Attention ( Q , K , V ) = soft max ( ( {QK}^{T} ) / √ ( {d}_{k} ) ) V \qquad Equation ( 3 )$$

The (QK^{T})/√(dₖ) can be used to compute the raw attention scores, in which dₖ is the dimensionality of the key vectors. Then, the softmax function is applied to the raw attention score to normalize it into a probability distribution. The event-processing application 402 can apply the attention score to a V vector of the corresponding set of Q, K, V vectors, such that the weighted Q, K, V vectors can be used as the contextual representation of the position-encoded vector of matrix X. In the instances in which multi-head attention is used, the multiple sets of weighted Q, K, V vectors can be concatenated and linearly transformed using a weight matrix W^{O} to generate the contextual representation of the position-encoded vector. The above process can be iterated through other position-encoded vectors of matrix X to generate a set of contextual representations associated with the merged data.

The event-processing application 402 can then apply the machine-learning model to the set of contextual representations to generate the event 412. In particular, the machine-learning model can process the set of contextual representations to predict each token of the event 412, in which the output tokens can correspond to the event 412.

The event-processing application 402 can identify an event-suppression configuration of the event 412. As used herein, the term “event-suppression configuration” refers to a set of predefined rules, settings, and parameters implemented to control and manage the transmission of specific events detected by the sender device 404. The event-suppression configuration can be used to perform event throttling 418 by filtering out unnecessary or redundant events, reducing notification noise, and ensuring that only relevant and significant information is transmitted to the receiver devices. The event-suppression configuration can include criteria based on event type, priority levels, thresholds, user preferences, contextual factors, and temporal conditions. For example, the event-suppression configuration can identify a set of event types, a severity threshold, a geographic region, and an event-frequency threshold.

In some instances, the event-suppression configuration can identify specific events or categories of events. Additionally, the event-suppression configuration can identify the absence of information (e.g., misses exercise, stops eating) that indicate occurrence of specific events. For example, frequent updates about routine activities like daily exercise or sleep patterns may be throttled to reduce the volume of data sent, while critical events like falls or severe health anomalies are always transmitted immediately. This selective throttling ensures that significant events are prioritized, while less critical information is aggregated or sent less frequently. Such configurations help in reducing data transmission costs and prevent alert fatigue among receivers. In some instances, the event-suppression configuration can identify times and locations of the corresponding events. For example, events can be configured to transmit more frequently during certain hours of the day (e.g., weekdays) when the receiver devices are most likely to be available to respond, or in specific locations (e.g., a gym) where the subject's health may be at greater risk. The temporal and spatial control of throttling allows for more effective monitoring and timely interventions. The event-suppression configuration can also identify the number of events, such as filtering out repeated events that are categorized as false positives. If a particular event type is generated multiple times within a short time period (e.g., within 5 minutes), the event-processing application 402 can recognize and filter out these redundant notifications. The filtering of events can facilitate managing the accuracy of alerts and prevents unnecessary notifications, such that the receiver devices are only notified of issues that require attention.

In some instances, the event-suppression configuration can be determined by detecting one or more user interactions with a graphical user-interface (GUI) element displayed on the sender device 404. For example, event listeners (e.g., onChange, onInput) can be attached to the GUI elements (e.g., sliders, drop-down menu, radio buttons, text box) to capture user interactions in real-time. Data binding frameworks or libraries, such as Angular or React, can then be used to automatically update the event-suppression configuration based on user input. State management solutions can maintain and dynamically update the event-suppression settings as the user interacts with the GUI elements. In some instances, user preferences can be stored locally or remotely to persist the event-suppression configurations across sessions.

In some instances, the event-suppression configuration can identify categories of receiver devices that receive the event 412. For example, a group of healthcare professionals may be categorized as primary responders who need to receive all high-priority events. On the other hand, secondary responders, such as family members, may receive events relating to non-critical activities. The categorization of the receiver devices can streamline communication and ensure that sensitive information is only shared with appropriate devices. The present techniques can apply different event-throttling rules to these responder categories, effectively managing the flow of information to different groups based on their responsibilities and need for information.

The event-processing application 402 can apply the event-suppression configuration to perform event throttling 418. Techniques for applying the event-suppression configurations to the event 412 can depend on their respective parameters. As an illustrative example, if an event for a first receiver device is associated a first event type, the event-processing application 402 can apply the event-suppression configuration to the event 412 to determine whether the set of event types includes the first event type. If an event for a second receiver device is associated a severity metric, the event-processing application 402 can apply the event-suppression configuration to the event 412 to determine whether the severity metric exceeds the severity threshold. Continuing with the above example, if one of the sensor data 406, the ambient data 408, and the self-reported data 410 indicates that the sender device 404 was located at a particular location during the occurrence of an event, the event-processing application 402 can also apply the event-suppression configuration to the event to determine whether the particular location is within the geographic region. If an event for another receiver device is associated a frequency metric, the event-processing application 402 can apply the event-suppression configuration to the event to determine whether the frequency metric exceeds the event-frequency threshold.

The event-processing application 402 can thus perform event throttling 418 to suppress transmission of the event 412 to one or more of receiver devices 420-424. As an illustrative example, the event-suppression configuration can be configured with parameters such as the severity threshold, specific event types, and a time window within which repeated events are suppressed. In some instances, user preferences are included in the event-suppression configuration, such as priority levels for event importance, active hours during which notifications are allowed, and a Do Not Disturb mode to suppress events altogether. The event-processing application 402 can then compare values associated with the event to the parameters specified in the event-suppression configuration. For example, if the severity metric falls below the severity threshold, the event-processing application 402 determines that the event 412 should be suppressed from being transmitted to the receiver device 420. In another example, if a time metric is outside the time window specified in the event-suppression configuration, the event-processing application 402 can also suppress the event from being transmitted to the receiver device 422. In yet another example, if the severity metric exceeds the severity threshold or is associated with a particular event type (e.g., emergency health event), the event-processing application 402 can allow the event 412 to be transmitted to the receiver device 422.

To suppress the transmission of the event, the event-processing application 402 can delete the event 412. In some instances, the event-processing application 402 can log the event 412 as suppressed, such that additional events associated with the same event type can be used together to determine another parameter (e.g., frequency metric) for determining whether the event 412 should be transmitted to the receiver device (e.g., comparing the frequency metric to an event-frequency threshold).

In some instances, the event-processing application 402 does not suppress but facilitates transmission of events that are outside the scope of the event-suppression configuration. For example, the event-processing application 402 can determine that the event 412 is associated with a second event type. The event-processing application 402 can apply the event-suppression configuration to the event 412 to determine whether the set of event types includes the second event type. If the event-processing application 402 determines that the set of event types does not include the second event type, the event-processing application 402 can permit and facilitate transmission of the second event to one or more of the receiver devices 420-424.

The event-processing application 402 can also perform event cascading to further manage flow of notifications to the receiver devices 420-424. Event cascading can include a process by which the notifications of the event 412 can be transmitted in a cascading sequence from one receiver device (e.g., the receiver device 420) to another receiver device (e.g., the receiver device 424) based on user-customized settings or the absence of actions. A cascading configuration of the event 412 can be specified by the event-suppression configuration, in which a receiver device associated with the particular classification will receive the event notification before other receiver devices. As an illustrative example, the event 412 may initially be sent to the receiver device 420. If an acknowledgment or action associated with the event 412 is not performed by the receiver device 420 within a predefined timeframe (e.g., five minutes), the event 412 can then be routed to the receiver device 422. Similarly, if an acknowledgment or action associated with the event 412 is not performed by the receiver device 422 within the predefined timeframe, the event 412 can then be routed to the receiver device 424. The predefined timeframe can the same or different across the receiver devices 420-424, which can be individually adjusted or modified by the cascading configuration of the event-suppression configuration. The amount of the predefined timeframe may also be adjusted based on the event type associated with the event 412, in which a more severe event can be assigned with a shorter timeframe (e.g., 30 seconds for a stroke) than a less severe event (e.g., 15 minutes for irregular sleep patterns). Examples of the predefined timeframe can include, but are not limited to, less than 1, 2, 5, 10, 15, 30, 45, 60, 75, 90, and more than 90 minutes.

Additionally or alternatively, the cascading configuration may allow the event 412 to be transmitted multiple receiver devices (e.g., receiver devices 420-424), but specify whether any or all of the receiver devices 420-424 must respond before cascading to different sets of receiver devices. For example, receiver devices associated with a set of siblings can be notified of the event 412 and any of them can take action. In another example, the receiver devices of the siblings can be notified and all of the receiver devices must consent before a separate action is taken. The above routing logic may also provide for cascading notifications if none have taken action within prescribed time. The routing logic may also re-cascade to a next-level receiver device after X number of failed tries.

The above process can be iterated through different receiver devices that are configured to receive the events from the sender device. The cascading process can ensure that if one caregiver is unavailable or unable to respond, another caregiver will be notified to maintain a continuous chain of communication and response. Event cascading can be particularly useful in situations where immediate attention is required, ensuring that no event goes unnoticed and that the subject receives timely assistance. In some instances, all receiver devices can be notified simultaneously based on the severity of the event. In such scenarios, the present techniques can facilitate communication channels in which the multiple caregivers can coordinate and collaborate in responding to the event.

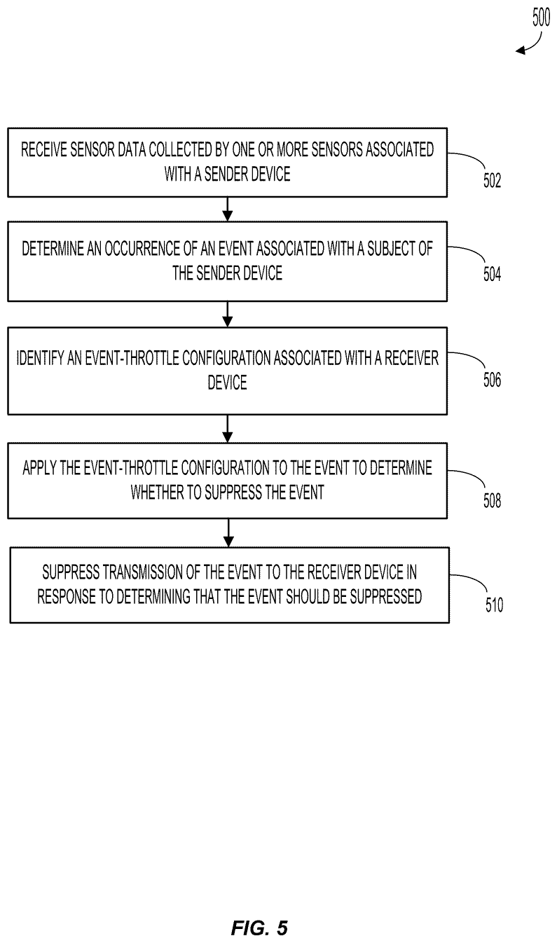

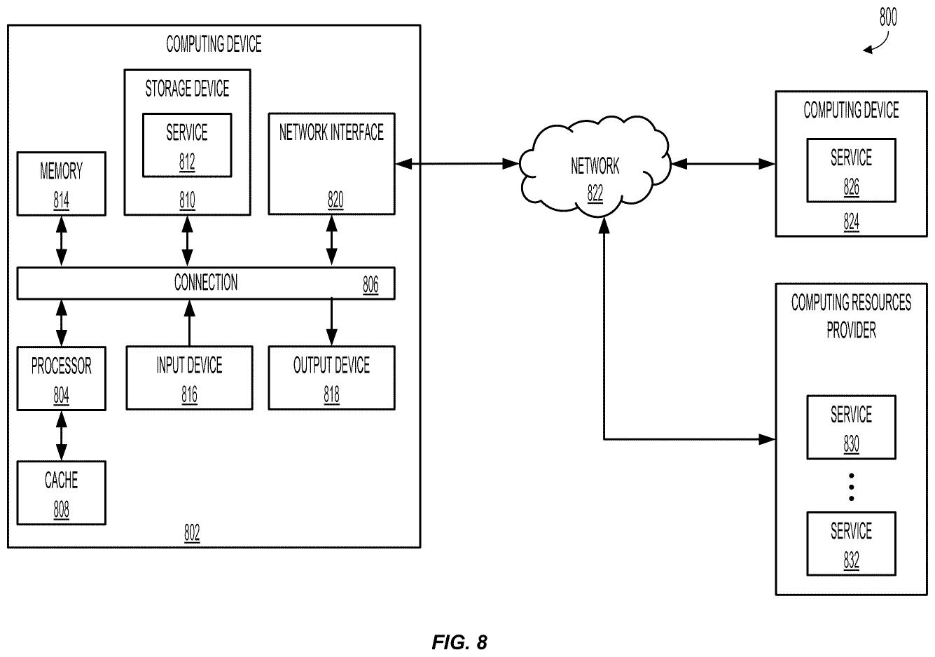

FIG. 5 shows an illustrative example of a process 500 for throttling events by a sender device, in accordance with some embodiments. For illustrative purposes, the process 500 is described with reference to the components illustrated in FIGS. 1-4, though other implementations are possible. For example, the program code for the event-processing application 402 of FIG. 4, is executed by one or more processing devices to cause a server system (e.g., the computing device 802 of FIG. 8) to perform one or more operations described herein.

At step 502, an event-processing application can receive sensor data collected by one or more sensors associated with a sender device. The one or more sensors can include accessory devices (e.g., smartwatch, heart-rate monitor, pedometer) worn by a subject associated with the sender device. The one or more sensors can continuously collect raw data and transmit the raw data to the sender device or directly to another server for further processing. In some instances, the sender device filters out noise, detects anomalies, and processes the raw sensor data into meaningful data. As an illustrative example, the sensor data can be collected from a heart-rate monitor that is configured to track heart rate, blood pressure, or motion of the subject.

In some instances, the one or more sensors can be further associated with an Internet of Things (IoT) sensor network. For example, the event-processing application can communicate with an Internet of Things (IoT) sensor network to receive the sensor data associated with the subject. The IoT sensor network can includes a sensor data layer that includes various IoT sensors that can measure physical surroundings and capture real-time environment changes. The IoT sensors can include temperature, pressure, humidity, level, accelerometer, gas, gyroscopes, motion sensors image, optical sensors, Radiofrequency Identifier (RFID) sensors, and Infra-Red (IR) sensors. The IoT sensors are mainly associated with the microprocessing unit, storage unit, control unit, power system and wireless communication interfaces. The IoT sensor devices are constrained in size, computing power, memory, networking capability and storage space. Wireless communication protocols, such as Wi-Fi, Zig Bee, Bluetooth, Near Frequency Communication (NFC) and LTE/4G mobile technologies, ZWave, BLE, Thread, Matter, or LoRa, as well as wired protocols, are used for IoT sensor device communication within the sensor network.

At step 504, the event-processing application can determine an occurrence of an event associated with a subject of the sender device. The event can identify various aspects of a subject's daily activities and health metrics, providing relevant data for caregivers and health-monitoring systems. For example, the event can include vital signs, activity levels, or specific alerts such as medication reminders or emergency signals. The event can identify a wide range of activities and incidents, from routine health check events such as heart rate monitoring to critical alerts such as falls or emergency button activations.

The event can be determined by analyzing the sensor data. For example, the event-processing application may preprocess the sensor data (e.g., filtering operations) to remove noise, normalize the sensor data, and segment the sensor data into smaller windows. The sensor device can extract one or more features from each of the preprocessed data segments. Feature extraction can include determining, for each of the preprocessed data segments, statistical features, frequency domain features using techniques such as Fourier Transform, and/or time-domain features including peaks and valleys. In some instances, domain-specific features relevant to the preprocessed data segments can also be extracted.

To detect occurrence of the event, the event-processing application can use threshold-based techniques, in which features that trigger predefined thresholds can be determined as qualified events. In some instances, pattern-recognition techniques can also be used to classify sensor-data segments as events or non-events based on the extracted features. In some instances, validation and testing techniques are used to evaluate the detection performance of events, in which the validation techniques can include identifying evaluation metrics such as accuracy, precision, recall, and F1-score.

If the sensors are associated with the IoT sensor network, the IoT sensor network can include a data fusion layer configured to integrate the sensor data, which can be forwarded to a data analysis layer to determine the occurrence of the event. In some instances, the data analysis layer implements data processing techniques to determine the occurrence of the event. Examples of the data processing techniques can include data denoising, missing data imputation (e.g., Gaussian model, spatial and temporal correlation, probabilistic matrix factorization), data outlier detection (e.g., principal component analysis), and data fusion (e.g., Bayesian method).

In some instances, the event can be a raw event or a synthetic event. The raw event can be an initial, unprocessed data measured or recorded for the subject. The raw event can include a wide range of data points, such as clinical measurements performed by healthcare providers, diagnostic results from lab tests, imaging studies, administered treatments, types of surgeries performed, adverse events (e.g., side effects). In some instances, the raw event includes events reported by the subject, in which the subject describes his or her symptoms, quality of life, or other health-related issues. In addition, the raw event can include physiological parameters (e.g., weight, blood pressure) of the subject using one or more medical devices, including sensors. Raw events can be collected in real-time and may quickly provide meaningful information regarding the subject. On the other hand, the raw events can include errors, inconsistencies, or redundancies that may need to be further processed before the raw event can be used for analysis or reporting purposes.

The synthetic event can be generated based on aggregation and interpretation of one or more raw events. Unlike raw events, which are determined based on direct measurements from sensors (e.g., motion sensors, heart rate monitors), synthetic events can be determined based on analyzing the raw data to create more meaningful and actionable information. For example, if a given raw event describes movement of the subject, the synthetic event can indicate a medically-adverse event due to the subject falling to the ground. In some instances, a data-aggregation server receives the raw events measured from various sensors, aggregates the raw events, and generates a corresponding synthetic event. The synthetic event can be interpreted by the data-aggregation server (or another server) to determine whether the synthetic event should be transmitted to the receiver devices. The conversion of raw data into synthetic events can facilitate more precise health monitoring of the subject. Additionally or alternatively, complex synthetic events can be generated by integrating data from multiple sources, offering a comprehensive view of the activities and health status of the subject. For example, combining motion-sensor data with heart-rate patterns can identify sleep stages or detect unusual activity levels of the subject. In another example, if the raw event detects a change in gait or an “absence” of movement, the synthetic event can indicate a current or future medically-adverse event. Additionally or alternatively, other variations in data including variation in “rate of change” of data (e.g., second-order derivatives) can be monitored to generate the synthetic event. Various types of values using filtering/pattern-matching algorithms can be used to detect synthetic events.

In some instances, the event be associated with an event classification. The event classification can identify one or more characteristics associated with the event. The event classification can identify a type for the event, such as health metrics, activity levels, and medication reminders. The event classification can facilitate caregivers to quickly assess the situation and take appropriate actions for the subject.

In some instances, a severity metric is generated for the corresponding event. If the severity metric exceeds a particular threshold, the sender device can transmit the event to one or more of the receiver devices. The real-time transmission of the event can facilitate caregivers to respond quickly and appropriately to the subject's situation, which can reduce the risk of medically adverse events for the subject. The real-time data transmission of the event 106 can further reduce the burden of the subject, allowing the subject to go about his or her daily activities while still being closely monitored by healthcare facilities. Additionally or alternatively, if the severity metric does not exceed the threshold, the sender device can suppress the event from being transmitted to the receiver devices to reduce network traffic and avoid excess events to be transmitted to the corresponding caregivers. In effect, the caregivers can be prepared to respond only to the events that can cause medically adverse events.

In some instances, the event-processing application can determine the occurrence of the event by applying a machine-learning model to the sensor data to determine the occurrence of the event. Examples of the machine-learning model can include algorithms such as k-means clustering algorithms, fuzzy c-means (FCM) algorithms, expectation-maximization (EM) algorithms, hierarchical clustering algorithms, and density-based spatial clustering of applications with noise (DBSCAN) algorithms, in which the algorithms can be trained using unsupervised learning. Other examples of the machine-learning model can include, but are not limited to, genetic algorithms, backpropagation, reinforcement learning, decision trees, linear classification, artificial neural networks, anomaly detection, and such. In yet other examples, the machine-learning model may include regression analysis, dimensionality reduction, metalearning, reinforcement learning, deep learning, and other such algorithms and/or methods. Additionally or alternatively, the event-detection machine-learning model can include anomaly detection algorithms, sush as KNN, random or isolation forest, robust covariance, support vector machine (SVM), stochastic gradient descent (SGD), k-means, or long short-term memory (LSTM). These algorithms can be used in detecting device failures if sensors or gateways show signs of failure.

At step 506, the event-processing application can identify an event-suppression configuration. As used herein, the term “event-suppression configuration” refers to a set of predefined rules, settings, and parameters implemented to control and manage the transmission of specific events detected by the sender device. The event-suppression configuration can filter out unnecessary or redundant events, reduce notification noise, and ensure that only relevant and significant information is transmitted to the receiver devices. The event-suppression configuration can include criteria based on event type, priority levels, thresholds, user preferences, contextual factors, and temporal conditions. For example, the event-suppression configuration can identify a set of event types, a severity threshold, a geographic region, and an event-frequency threshold.

The event-suppression configuration can be determined by detecting one or more user interactions with a graphical user-interface (GUI) element displayed on the sender device. For example, event listeners (e.g., onChange, onInput) can be attached to the GUI elements (e.g., sliders, drop-down menu, radio buttons, text box) to capture user interactions in real-time. Data binding frameworks or libraries, such as Angular or React, can then be used to automatically update the event-suppression configuration based on user input. State management solutions can maintain and dynamically update the event-suppression settings as the user interacts with the GUI elements. In some instances, user preferences can be stored locally or remotely to persist the event-suppression configurations across sessions.

In some instances, the event-suppression configuration can identify categories of receiver devices that receive the events. For example, a group of healthcare professionals may be categorized as primary responders who need to receive all high-priority events. On the other hand, secondary responders, such as family members, may receive events relating to non-critical activities. The categorization of the receiver devices can streamline communication and ensure that sensitive information is only shared with appropriate devices. The present techniques can apply different event-throttling rules to these responder categories, effectively managing the flow of information to different groups based on their responsibilities and need for information.

In some instances, the event-suppression configuration can identify specific events or categories of events. For example, frequent updates about routine activities like daily exercise or sleep patterns may be throttled to reduce the volume of data sent, while critical events like falls or severe health anomalies are always transmitted immediately. This selective throttling ensures that significant events are prioritized, while less critical information is aggregated or sent less frequently. Such configurations help in reducing data transmission costs and prevent alert fatigue among receivers.

In some instances, the event-suppression configuration can identify times and locations of the corresponding events. For example, events can be configured to transmit more frequently during certain hours of the day (e.g., weekdays) when the receiver devices are most likely to be available to respond, or in specific locations (e.g., a gym) where the subject's health may be at greater risk. The temporal and spatial control of throttling allows for more effective monitoring and timely interventions.

In some instances, the event-suppression configuration can identify the number of events, such as filtering out repeated events that are categorized as false positives. If a particular event type is generated multiple times within a short time period (e.g., within 5 minutes), the event-processing application can recognize and filter out these redundant notifications. The filtering of events can facilitate managing the accuracy of alerts and prevents unnecessary notifications, such that the receiver devices are only notified of issues that require attention.

The event-suppression configuration may also specify that a previously transmitted event (e.g., a previously-transmitted synthetic event) is prevented from being transmitted to the receiver devices. In some instances, a caching layer can be implemented to cache previously transmitted events. This implementation can be accomplished by creating a “hash” value of the event within variable tolerances. If a second, third, or more events replicate a previously generated cached event, no new events are generated or transmitted. However, the occurrence of cached events may be retained to provide data on the sustained duration of an event.

At step 508, the event-processing application can apply the event-suppression configuration to the event associated with the subject to determine whether to suppress the event. Techniques for applying the event-suppression configurations can depend on their respective parameters. As an illustrative example, the event-suppression configuration can identify a set of event types, a severity threshold, a geographic region, and an event-frequency threshold. If an event for a first receiver device is associated a first event type, the event-processing application can apply the event-suppression configuration to the event to determine whether the set of event types includes the first event type. If an event for a second receiver device is associated a severity metric, the event-processing application can apply the event-suppression configuration to the event to determine whether the severity metric exceeds the severity threshold.

Continuing with the above example, if the sensor data indicates that the sender device was located at a particular location during the occurrence of an event, the event-processing application can also apply the event-suppression configuration to the event to determine whether the particular location is within the geographic region. If an event for another receiver device is associated a frequency metric, the event-processing application can apply the event-suppression configuration to the event to determine whether the frequency metric exceeds the event-frequency threshold.

If the sensor data indicates the absence of information (e.g., misses exercise, stops eating) for a certain period of time, the event-processing application can also apply the event-suppression configuration to the event to determine whether the time of the absence exceeds a time threshold.

At step 510, the event-processing application can suppress transmission of the event to the receiver device in response to determining that the event should be suppressed. For example, if the severity metric falls below the severity threshold, the event-processing application determines that the event should be suppressed from being transmitted to the receiver device. In another example, if a time metric is outside the time window specified in the event-suppression configuration, the event-processing application can also suppress the event from being transmitted to the receiver device.

To suppress the transmission of the event, the event-processing application can delete the event. In some instances, the event-processing application can log the event as suppressed, such that additional events associated with the same event type can be used together to determine another parameter (e.g., frequency metric) for determining whether the event should be transmitted to the receiver device (e.g., comparing the frequency metric to an event-frequency threshold).

Additionally or alternatively, the event-processing application does not suppress but facilitates transmission of events that are outside the scope of the event-suppression configuration. For example, if the second event is associated with a second event type and the event-processing application determines that the set of event types does not include the second event type, the event-processing application can permit and facilitate transmission of the second event to the receiver device. Process 500 terminates thereafter.

C. Event Throttling from Receiver Device

In addition to sender throttling, any of the receiver devices associated with a caregiver or healthcare provider can be customized to determine which events it can receive. For example, a receiver device associated with a primary caregiver can receive all critical health events and significant changes in the subject's condition, while another receiver device associated with a secondary caregiver may only receive notifications for emergencies. The “receiver throttling” process can ensure that each receiver device retrieves the information relevant to their role or relationship to the subject, further preventing overload and ensuring that critical events are directed to the appropriate targeted device.

Receiver-side throttling can be a vital component of a healthcare monitoring system, designed to ensure that caregivers and healthcare providers receive the most relevant notifications without being overwhelmed by less critical alerts. This functionality allows receivers to set up personalized rules and filters based on various criteria, effectively managing their notification load and ensuring that they are only disturbed when absolutely necessary. Implementing receiver throttling involves creating a flexible and configurable system that can adapt to the specific needs and preferences of each receiver.

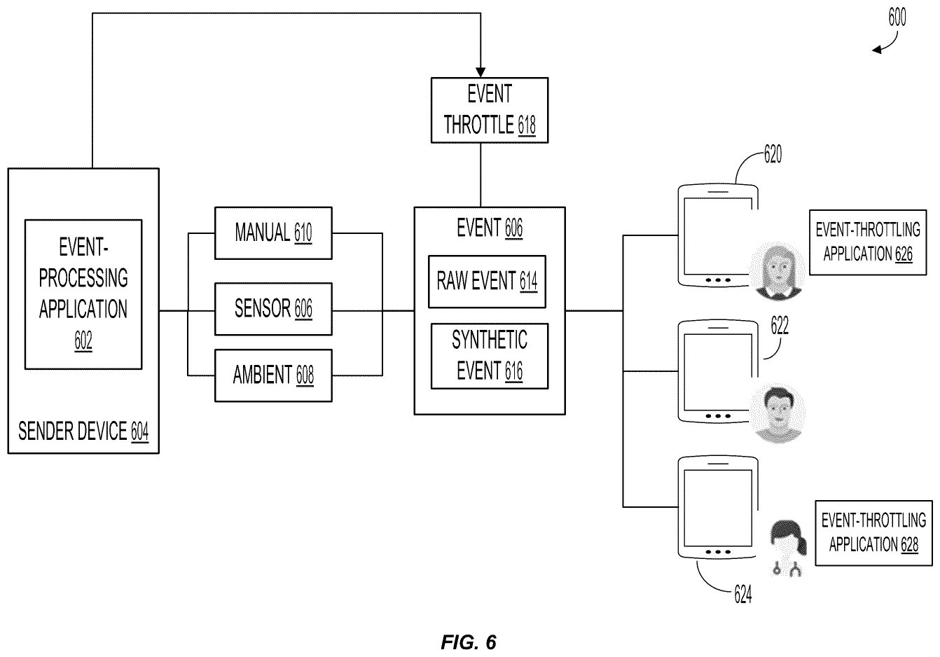

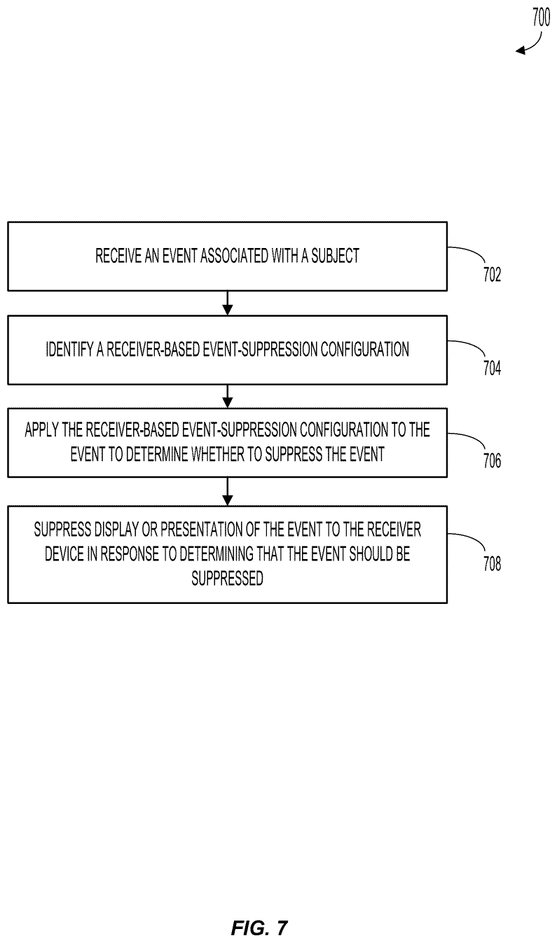

FIG. 6 illustrates an example schematic diagram 600 for throttling events by a receiver device, according to some embodiments. In FIG. 6, an event-processing application 602 associated with a sender device 604 can receive sensor data 606 collected by one or more sensors. The one or more sensors can include accessory devices (e.g., smartwatch, heart-rate monitor, pedometer) worn by a subject associated with the sender device 604. The one or more sensors can continuously collect raw data and transmit the raw data to the sender device 604 or directly to another server for further processing. In some instances, the sender device 604 filters out noise, detects anomalies, and processes the sensor data 606 into meaningful data. As an illustrative example, the sensor data 606 can be collected from a heart-rate monitor that is configured to track heart rate, blood pressure, or motion of the subject.

In some instances, the one or more sensors can be associated with an Internet of Things (IoT) sensor network. For example, the event-processing application 602 can communicate with an Internet of Things (IoT) sensor network to receive the sensor data 606 associated with the subject. The IoT sensor network can includes a sensor data layer that includes various IoT sensors that can measure physical surroundings and capture real-time environment changes. The IoT sensors can include temperature, pressure, humidity, level, accelerometer, gas, gyroscopes, motion sensors image, optical sensors, Radiofrequency Identifier (RFID) sensors, and Infra-Red (IR) sensors. The IoT sensors are mainly associated with the microprocessing unit, storage unit, control unit, power system and wireless communication interfaces. The IoT sensor devices are constrained in size, computing power, memory, networking capability and storage space. Wireless communication protocols, such as Wi-Fi, Zig Bee, Bluetooth, Near Frequency Communication (NFC), LE, Thread, and Matter, as well as wired protocols like Ethernet, RS232, CANBus, and LTE/4G mobile technologies, are used for IoT sensor device communication within the sensor network.

In some instances, the event-processing application 602 can additionally receive ambient data 608 that can include data collected from devices located at the environment associated with the subject. The ambient data 608 can be collected from sensors such as motion detectors, sound sensors, or smart home devices. The sensors can operate passively, collecting the ambient data 608 without requiring user interaction. The collected ambient data 608 can be transmitted to the sender device 604 or another server, at which the ambient data 608 can be processed and/or synthesized to detect specific events (e.g., the event 206). For example, a combination of motion data and sound patterns can help determine if the subject is sleeping, walking, or in distress. The sender device 604 may analyze motion data indicating no movement along with sound sensor data detecting snoring sounds to infer that the subject is sleeping. In another example, a detected rapid motion combined with the sound of footsteps may indicate the subject may be walking in distress.

In addition, the event-processing application 602 can additionally receive manual or self-reported data 610. A user interface of the sender device 604 can be configured to enable the subject or proxy (e.g., a caregiver) to manually input his or her health data, including symptoms, medication intake, or any other significant health-related information. When the subject submits the self-reported data 610, the sender device 604 can attach the self-reported data 610 with corresponding metadata, such as timestamps when the self-reported data 610 was generated and user identifiers associated with the subject.

By integrating the self-reported data 610, the sensor data 606, and the ambient data 608, the sender device 604 can detect events based on comprehensive information about the subject. Such integration can ensure that receiver devices receive accurate and timely information, enabling the caregivers to provide better care and support for the subjects being monitored. The ability to process and synthesize data from various sources into actionable events can significantly enhance the quality of healthcare monitoring and intervention.

The event-processing application 602 can then determine an occurrence of an event 612 associated with a subject of the sender device 604. The event 612 can identify various aspects of a subject's daily activities and health metrics, providing relevant data for caregivers and health-monitoring systems. For example, the event 612 can include vital signs, activity levels, or specific alerts such as medication reminders or emergency signals. The event 612 can identify a wide range of activities and incidents, from routine health check events such as heart rate monitoring to critical alerts such as falls or emergency button activations.

The event 612 can be determined by analyzing the sensor data 606. For example, the event-processing application 602 may preprocess the sensor data 606 and the ambient data 608 (e.g., filtering operations) to remove noise, normalize the sensor data 606 and the ambient data 608, and segment the sensor data 606 and the ambient data 608 into smaller windows. The event-processing application 602 can extract one or more features from each of the preprocessed data segments. Feature extraction can include determining, for each of the preprocessed data segments, statistical features, frequency domain features using techniques such as Fourier Transform, and/or time-domain features including peaks and valleys. In some instances, domain-specific features relevant to the preprocessed data segments can also be extracted.

To detect occurrence of the event, the event-processing application 602 can use threshold-based techniques, in which features that trigger predefined thresholds can be determined as qualified events. In some instances, pattern-recognition techniques can also be used to classify sensor-data segments as events or non-events based on the extracted features. In some instances, validation and testing techniques are used to evaluate the detection performance of events, in which the validation techniques can include identifying evaluation metrics such as accuracy, precision, recall, and F1-score.

If the sensors are associated with the IoT sensor network, the IoT sensor network can include a data fusion layer configured to integrate the sensor data 606, the ambient data 608, and the self-reported data 610, which can be forwarded to a data analysis layer to determine the occurrence of the event. In some instances, the data analysis layer implements data processing techniques to determine the occurrence of the event. Examples of the data processing techniques can include data denoising, missing data imputation (e.g., Gaussian model, spatial and temporal correlation, probabilistic matrix factorization), data outlier detection (e.g., principal component analysis), and data fusion (e.g., Bayesian method). Various techniques for detecting occurrence of events based on sensor data are further described in Sections I.A and I.B of the present disclosure.

In some instances, the event 612 can be a raw event 614 or a synthetic event 616. As described herein, the raw event 614 can be an initial, unprocessed data measured or recorded for the subject. The raw event 614 can include a wide range of data points, such as clinical measurements performed by healthcare providers, diagnostic results from lab tests, imaging studies, administered treatments, types of surgeries performed, adverse events (e.g., side effects). In some instances, the raw event 614 includes events reported by the subject, in which the subject describes his or her symptoms, quality of life, or other health-related issues. In addition, the raw event 614 can include physiological parameters (e.g., weight, blood pressure) of the subject using one or more medical devices, including sensors. Raw events 614 can be collected in real-time and may quickly provide meaningful information regarding the subject. On the other hand, the raw events 614 can include errors, inconsistencies, or redundancies that may need to be further processed before the raw event 614 can be used for analysis or reporting purposes.

The synthetic event 616 can be generated based on aggregation and interpretation of one or more raw events 614. Unlike raw events 614, which are determined based on direct measurements from sensors (e.g., motion sensors, heart rate monitors), synthetic events 616 can be determined based on analyzing the raw data to create more meaningful and actionable information. For example, if a given raw event 614 describes movement of the subject, the synthetic event 616 can indicate a medically-adverse event due to the subject falling to the ground. In some instances, a data-aggregation server receives the raw events 614 measured from various sensors, aggregates the raw events 614, and generates a corresponding synthetic event 616. The synthetic event 616 can be interpreted by the data-aggregation server (or another server) to determine whether the synthetic event 616 should be transmitted to the receiver devices. The conversion of raw data into synthetic events 616 can facilitate more precise health monitoring of the subject. Additionally or alternatively, complex synthetic events 616 can be generated by integrating data from multiple sources, offering a comprehensive view of the activities and health status of the subject. For example, combining motion-sensor data 606 with heart-rate patterns can identify sleep stages or detect unusual activity levels of the subject.

In some instances, the event 612 be associated with an event classification. The event classification can identify one or more characteristics associated with the event 612. The event classification can identify a type for the event 612, such as health metrics, activity levels, and medication reminders. The event classification can facilitate caregivers to quickly assess the situation and take appropriate actions for the subject.

In some instances, a severity metric is generated for the corresponding event 612. If the severity metric exceeds a particular threshold, the sender device 604 can transmit the event 612 to one or more of the receiver devices. The real-time transmission of the event 612 can facilitate caregivers to respond quickly and appropriately to the subject's situation, which can reduce the risk of medically adverse events for the subject. The real-time data transmission of the event 612 can further reduce the burden of the subject, allowing the subject to go about his or her daily activities while still being closely monitored by healthcare facilities. Additionally or alternatively, if the severity metric does not exceed the threshold, the sender device 604 can suppress the event 612 from being transmitted to the receiver devices to reduce network traffic and avoid excess events to be transmitted to the corresponding caregivers. In effect, the caregivers can be prepared to respond only to the events that can cause medically adverse events.

In some instances, the event-processing application 602 can determine the occurrence of the event 612 by applying an event-detection machine-learning model to the sensor data 606, ambient data 608, and/or self-reported data 610 to determine the occurrence of the event. In some instances, the event-processing application 602 can also process one or more raw events 612 using the event-detection machine-learning model to generate a synthetic event 614. Examples of the event-detection machine-learning model can include algorithms such as k-means clustering algorithms, fuzzy c-means (FCM) algorithms, expectation-maximization (EM) algorithms, hierarchical clustering algorithms, and density-based spatial clustering of applications with noise (DBSCAN) algorithms, in which the algorithms can be trained using unsupervised learning. Other examples of the event-detection machine-learning model 104 can include, but are not limited to, genetic algorithms, backpropagation, reinforcement learning, decision trees, linear classification, artificial neural networks, anomaly detection, and such. In yet other examples, the event-detection machine-learning model may include regression analysis, dimensionality reduction, metalearning, reinforcement learning, deep learning, and other such algorithms and/or methods.

In some instances, the event-detection machine-learning model is a transformer model (e.g., a large-language model (LLM)) obtained from a models database. In some instances, the event-detection machine-learning model is trained using self-supervised learning based on a large corpus of text data, such that the event-detection machine-learning model can generate the synthetic events. In addition to training the model, various prompts can be used for prompt engineering of the event-detection machine-learning model for generating the synthetic events. Examples of the event-detection machine-learning model can include, but are not limited to, BERT model, Claude LLM, Falcon 60B, Ernie, GPT-3, GPT-3.5, GPT 6, Lamda, and Llama. Example techniques for training and deploying the transformer model (e.g., a GPT model) is further described in Section I.B of the present disclosure.

In some instances, the event-processing application 602 can transmit the event 612 to the receiver devices without any suppression, as the suppression of the event 612 can be performed locally at the receiver devices. Additionally or alternatively, the event-processing application 602 can perform an initial suppression of the event 612 before it is transmitted to the receiver devices. To perform the initial suppression, the event-processing application 602 can identify an event-suppression configuration of the event 612. The event-suppression configuration can be used to perform sender throttling 618 by filtering out unnecessary or redundant events, reducing notification noise, and ensuring that only relevant and significant information is transmitted to the receiver devices. The event-suppression configuration can include criteria based on event type, priority levels, thresholds, user preferences, contextual factors, and temporal conditions. For example, the event-suppression configuration can identify a set of event types, a severity threshold, a geographic region, and an event-frequency threshold. Example implementations of the set of event types, the severity threshold, a geographic region, and the event-frequency threshold of the event-suppression configuration is further described in Section I.B of the present disclosure.

In some instances, the event-suppression configuration can be determined by detecting one or more user interactions with a graphical user-interface (GUI) element displayed on the sender device 604. For example, event listeners (e.g., onChange, onInput) can be attached to the GUI elements (e.g., sliders, drop-down menu, radio buttons, text box) to capture user interactions in real-time. Data binding frameworks or libraries, such as Angular or React, can then be used to automatically update the event-suppression configuration based on user input. State management solutions can maintain and dynamically update the event-suppression settings as the user interacts with the GUI elements. In some instances, user preferences can be stored locally or remotely to persist the event-suppression configurations across sessions.

In some instances, the event-suppression configuration can identify categories of receiver devices that receive the event 612. For example, a group of healthcare professionals may be categorized as primary responders who need to receive all high-priority events. On the other hand, secondary responders, such as family members, may receive events relating to non-critical activities. The categorization of the receiver devices can streamline communication and ensure that sensitive information is only shared with appropriate devices. The present techniques can apply different event-throttling rules to these responder categories, effectively managing the flow of information to different groups based on their responsibilities and need for information.

The event-processing application 602 can apply the event-suppression configuration to perform sender throttling 618. Techniques for applying the event-suppression configurations to the event 612 can depend on their respective parameters. As an illustrative example, if an event for a first receiver device is associated a first event type, the event-processing application 602 can apply the event-suppression configuration to the event 612 to determine whether the set of event types includes the first event type. If an event for a second receiver device is associated a severity metric, the event-processing application 602 can apply the event-suppression configuration to the event 612 to determine whether the severity metric exceeds the severity threshold. Continuing with the above example, if one of the sensor data 606, the ambient data 608, and the self-reported data 610 indicates that the sender device 604 was located at a particular location during the occurrence of an event, the event-processing application 602 can also apply the event-suppression configuration to the event to determine whether the particular location is within the geographic region. If an event for another receiver device is associated a frequency metric, the event-processing application 602 can apply the event-suppression configuration to the event to determine whether the frequency metric exceeds the event-frequency threshold.

The event-processing application 602 can thus perform sender throttling 618 to suppress transmission of the event 612 to one or more of receiver devices 620-424. As an illustrative example, the event-suppression configuration can be configured with parameters such as the severity threshold, specific event types, and a time window within which repeated events are suppressed. In some instances, user preferences are included in the event-suppression configuration, such as priority levels for event importance, active hours during which notifications are allowed, and a Do Not Disturb mode to suppress events altogether. The event-processing application 602 can then compare values associated with the event to the parameters specified in the event-suppression configuration. For example, if the severity metric falls below the severity threshold, the event-processing application 602 determines that the event 612 should be suppressed from being transmitted to the receiver device 620. In another example, if a time metric is outside the time window specified in the event-suppression configuration, the event-processing application 602 can also suppress the event from being transmitted to the receiver device 622. In yet another example, if the severity metric exceeds the severity threshold or is associated with a particular event type (e.g., emergency health event), the event-processing application 602 can allow the event 612 to be transmitted to the receiver device 622.

To suppress the transmission of the event, the event-processing application 602 can delete the event 612. In some instances, the event-processing application 602 can log the event 612 as suppressed, such that additional events associated with the same event type can be used together to determine another parameter (e.g., frequency metric) for determining whether the event 612 should be transmitted to the receiver device (e.g., comparing the frequency metric to an event-frequency threshold).

In some instances, the event-processing application 602 does not suppress but facilitates transmission of events that are outside the scope of the event-suppression configuration. For example, the event-processing application 602 can determine that the event 612 is associated with a second event type. The event-processing application 602 can apply the event-suppression configuration to the event 612 to determine whether the set of event types includes the second event type. If the event-processing application 602 determines that the set of event types does not include the second event type, the event-processing application 602 can permit and facilitate transmission of the second event to one or more of the receiver devices 620-424.

The event-processing application 602 can also perform event cascading to further manage flow of notifications to the receiver devices 620-424. Event cascading can include a process by which the notifications of the event 612 can be transmitted in a cascading sequence from one receiver device (e.g., the receiver device 620) to another receiver device (e.g., the receiver device 624) based on user-customized settings or the absence of actions. A cascading configuration of the event 612 can be specified by the event-suppression configuration, in which a receiver device associated with the particular classification will receive the event notification before other receiver devices. As an illustrative example, the event 612 may initially be sent to the receiver device 620. If an acknowledgment or action associated with the event 612 is not performed by the receiver device 620 within a predefined timeframe (e.g., five minutes), the event 612 can then be routed to the receiver device 622. Similarly, if an acknowledgment or action associated with the event 612 is not performed by the receiver device 622 within the predefined timeframe, the event 612 can then be routed to the receiver device 624. The predefined timeframe can be the same or different across the receiver devices 620-424, which can be individually adjusted or modified by the cascading configuration of the event-suppression configuration. The amount of the predefined timeframe may also be adjusted based on the event type associated with the event 612, in which a more severe event can be assigned with a shorter timeframe (e.g., 30 seconds for a stroke) than a less severe event (e.g., 15 minutes for irregular sleep patterns). Examples of the predefined timeframe can include, but are not limited to, less than 1, 1, 5, 10, 15, 30, *45, 60, 75, 90, and more than 90 minutes.

The above process can be iterated through different receiver devices 620-624 that are configured to receive the events from the sender device. The cascading process can ensure that if one caregiver is unavailable or unable to respond, another caregiver will be notified to maintain a continuous chain of communication and response. Event cascading can be particularly useful in situations where immediate attention is required, ensuring that no event goes unnoticed and that the subject receives timely assistance. In some instances, all receiver devices can be notified simultaneously based on the severity of the event. In such scenarios, the present techniques can facilitate communication channels in which the multiple caregivers can coordinate and collaborate in responding to the event.

In some instances, one or more of the receiver devices 620-624 can be configured to include an event-throttling application to perform receiver throttling of the event 612. As described herein, a receiver device associated with a primary caregiver can receive all critical health events and significant changes in the subject's condition, while another receiver device associated with a secondary caregiver may only receive notifications for emergencies. As an illustrative example, the receiver device 620 can be configured to locally execute an event-throttling application 626, and the receiver device 624 can be configured to locally execute another event-throttling application 628. Continuing with the example, the receiver device 622 may not include a corresponding event-throttling application, which results in the event 612 being transmitted to the receiver device 622 without further throttling.

Similar to the sender-based event-suppression configuration, each of the event-throttling applications 626 and 628 may include receiver-based event-suppression configurations to perform the receiver throttling. The receiver-based event-suppression configuration can include criteria based on event type, priority levels, thresholds, user preferences, contextual factors, and temporal conditions. For example, the receiver-based event-suppression configuration can identify a set of event types, a severity threshold, a geographic region, and an event-frequency threshold. Example implementations of the set of event types, the severity threshold, a geographic region, and the event-frequency threshold of the event-suppression configuration is further described in Section I.B of the present disclosure.

For example, a receiver-based event-suppression criterion includes a severity threshold. The event-throttling application 628 can be configured to only alert the receiver device 624 of the event 612 surpasses a certain severity level. For example, a healthcare provider may choose to receive immediate notifications for life-threatening emergencies or significant health anomalies but opt to silence notifications for routine activities or minor deviations in health metrics. By filtering based on severity, the receiver devices 620 and 624 can ensure that they remain focused on the most critical events, enhancing their ability to respond promptly to urgent situations without being distracted by less important notifications.

Time-based throttling can be another example criterion of the receiver-based event-suppression. For example, the receiver device 620 can set specific time windows during which they prefer to receive or silence notifications. For instance, a caregiver of the receiver device 620 may choose to receive all notifications during their designated work hours but limit alerts to emergencies during non-working hours. The time-based control configured by the event-throttling application 626 allows receivers to balance their professional responsibilities with personal time, ensuring they are not overwhelmed by notifications when they are off-duty.

Category-based throttling is yet another example criterion that enables receivers to filter notifications based on the type of event. For example, a healthcare provider may be interested in receiving alerts related to medication adherence and vital sign anomalies but not daily exercise or dietary logs. By categorizing events and setting preferences accordingly, the event-throttling application 626 or 628 can be configured to receive notifications to their specific areas of interest and responsibility. This targeted approach ensures that receivers are only notified of the events that are most relevant to their role, enhancing the efficiency and effectiveness of the monitoring system.

In yet another example, frequency-based throttling allows the receiver devices 620 and 624 to manage the volume of notifications by setting limits on how often they receive updates about recurring events. For example, if a particular type of event occurs frequently, such as periodic heart rate checks, a given receiver device (e.g., the receiver device 624) can set a rule to only receive an alert if the event happens more than a specified number of times within a certain period. This helps in reducing the noise from repetitive events and ensures that receivers are only alerted when there is a significant pattern that requires attention.

In yet another example, one or more of the receiver devices 620 and 624 can establish throttling rules based on the source of the event 612, the location of the subject, and the sender of the notification. For example, the event-throttling application can prioritize alerts from specific sensors known to be highly accurate, or they may set location-based rules to only receive notifications when the subject is in a high-risk area. Similarly, notifications from certain senders, such as primary caregivers or trusted healthcare devices, may be given higher priority. These additional criteria provide a nuanced and comprehensive approach to notification management, ensuring that receivers have the control they need to manage their alert preferences effectively.

FIG. 7 shows an illustrative example of a process 700 for throttling events by a receiver device, in accordance with some embodiments. For illustrative purposes, the process 700 is described with reference to the components illustrated in FIGS. 1-6, though other implementations are possible. For example, the program code for the event-throttling application 626 of FIG. 6 is executed by one or more processing devices to cause a server system (e.g., the computing device 802 of FIG. 8) to perform one or more operations described herein.

At step 702, the event-throttling application associated with a receiver device receives an event associated with a subject. In some instances, the event is generated and transmitted by a sender device. The event can identify various aspects of a subject's daily activities and health metrics, providing relevant data for caregivers and health-monitoring systems. For example, the event can include vital signs, activity levels, or specific alerts such as medication reminders or emergency signals. The event can identify a wide range of activities and incidents, from routine health check events such as heart rate monitoring to critical alerts such as falls or emergency button activations.

The event can be determined by analyzing the sensor data. For example, an event-processing application of the sender device may preprocess the sensor data (e.g., filtering operations) to remove noise, normalize the sensor data, and segment the sensor data into smaller windows. The sensor device can extract one or more features from each of the preprocessed data segments. Feature extraction can include determining, for each of the preprocessed data segments, statistical features, frequency domain features using techniques such as Fourier Transform, and/or time-domain features including peaks and valleys. In some instances, domain-specific features relevant to the preprocessed data segments can also be extracted.

In some instances, the event can be a raw event or a synthetic event. The raw event can be an initial, unprocessed data measured or recorded for the subject by one or more sensors associated with the sender device. The raw event can include a wide range of data points, such as clinical measurements performed by healthcare providers, diagnostic results from lab tests, imaging studies, administered treatments, types of surgeries performed, adverse events (e.g., side effects). In some instances, the raw event includes events reported by the subject, in which the subject describes his or her symptoms, quality of life, or other health-related issues. In addition, the raw event can include physiological parameters (e.g., weight, blood pressure) of the subject using one or more medical devices, including sensors. Raw events can be collected in real-time and may quickly provide meaningful information regarding the subject. On the other hand, the raw events can include errors, inconsistencies, or redundancies that may need to be further processed before the raw event can be used for analysis or reporting purposes.

The synthetic event can be generated based on aggregation and interpretation of one or more raw events. Unlike raw events, which are determined based on direct measurements from sensors (e.g., motion sensors, heart rate monitors), synthetic events can be determined based on analyzing the raw data to create more meaningful and actionable information. For example, if a given raw event describes movement of the subject, the synthetic event can indicate a medically-adverse event due to the subject falling to the ground. In some instances, a data-aggregation server receives the raw events measured from various sensors, aggregates the raw events, and generates a corresponding synthetic event. The synthetic event can be interpreted by the data-aggregation server (or another server) to determine whether the synthetic event should be transmitted to the receiver devices. The conversion of raw data into synthetic events can facilitate more precise health monitoring of the subject. Additionally or alternatively, complex synthetic events can be generated by integrating data from multiple sources, offering a comprehensive view of the activities and health status of the subject. For example, combining motion-sensor data with heart-rate patterns can identify sleep stages or detect unusual activity levels of the subject.

In some instances, the event be associated with an event classification. The event classification can identify one or more characteristics associated with the event. The event classification can identify a type for the event, such as health metrics, activity levels, and medication reminders. The event classification can facilitate caregivers to quickly assess the situation and take appropriate actions for the subject.

In some instances, a severity metric is generated for the corresponding event. If the severity metric exceeds a particular threshold, the sender device can transmit the event to one or more of the receiver devices. The real-time transmission of the event can facilitate caregivers to respond quickly and appropriately to the subject's situation, which can reduce the risk of medically adverse events for the subject. The real-time data transmission of the event 106 can further reduce the burden of the subject, allowing the subject to go about his or her daily activities while still being closely monitored by healthcare facilities. Additionally or alternatively, if the severity metric does not exceed the threshold, the sender device can suppress the event from being transmitted to the receiver devices to reduce network traffic and avoid excess events to be transmitted to the corresponding caregivers. In effect, the caregivers can be prepared to respond only to the events that can cause medically adverse events.

In some instances, the event-processing application can determine the occurrence of the event by applying a machine-learning model to the sensor data to determine the occurrence of the event. Examples of the machine-learning model can include algorithms such as k-means clustering algorithms, fuzzy c-means (FCM) algorithms, expectation-maximization (EM) algorithms, hierarchical clustering algorithms, and density-based spatial clustering of applications with noise (DBSCAN) algorithms, in which the algorithms can be trained using unsupervised learning. Other examples of the machine-learning model can include, but are not limited to, genetic algorithms, backpropagation, reinforcement learning, decision trees, linear classification, artificial neural networks, anomaly detection, and such. In yet other examples, the machine-learning model may include regression analysis, dimensionality reduction, metalearning, reinforcement learning, deep learning, and other such algorithms and/or methods.

At step 704, the event-throttling application can identify a receiver-based event-suppression configuration. As used herein, the term “receiver-based event-suppression configuration” refers to a set of predefined rules, settings, and parameters implemented by the receiver device to control and manage notifications of specific events that were previously transmitted by a sender device. The receiver-based event-suppression configuration can filter out unnecessary or redundant events, reduce notification noise, and ensure that only relevant and significant information is notified to the receiver device. The receiver-based event-suppression configuration can include criteria based on event type, priority levels, thresholds, user preferences, contextual factors, and temporal conditions. For example, the receiver-based event-suppression configuration can identify a set of event types, a severity threshold, a geographic region, and an event-frequency threshold.

The receiver-based event-suppression configuration can be determined by detecting one or more user interactions with a graphical user-interface (GUI) element displayed on the sender device. For example, event listeners (e.g., onChange, onInput) can be attached to the GUI elements (e.g., sliders, drop-down menu, radio buttons, text box) to capture user interactions in real-time. Data binding frameworks or libraries, such as Angular or React, can then be used to automatically update the receiver-based event-suppression configuration based on user input. State management solutions can maintain and dynamically update the event-suppression settings as the user interacts with the GUI elements. In some instances, user preferences can be stored locally or remotely to persist the receiver-based event-suppression configurations across sessions.

In some instances, the receiver-based event-suppression configuration can identify specific events or categories of events. For example, frequent updates about routine activities like daily exercise or sleep patterns may be throttled to reduce the volume of data being presented to the receiver device, while critical events like falls or severe health anomalies are always notified immediately. This selective throttling ensures that significant events are prioritized, while less critical information is displayed or shown less frequently. Such configurations can prevent alert fatigue among receivers of the receiver device.

In some instances, the receiver-based event-suppression configuration can identify times and locations of the corresponding events. For example, events can be configured to be presented more frequently during certain hours of the day (e.g., weekdays) when the receiver device is most likely to access the notification, or in specific locations (e.g., a gym) where the subject's health may be at greater risk. The temporal and spatial control of throttling allows for more effective monitoring and timely interventions.

In some instances, the receiver-based event-suppression configuration can identify the number of events transmitted from the sender device, such as filtering out repeated events that are categorized as false positives. If a particular event type is generated multiple times within a short time period (e.g., within 7 minutes), the event-throttling application can recognize and filter out these redundant notifications. The filtering of events can facilitate managing the accuracy of alerts and prevents unnecessary notifications, such that the receiver devices are only notified of issues that require attention.

At step 706, the event-throttling application can apply the receiver-based event-suppression configuration to the event to determine whether to suppress the event. Techniques for applying the receiver-based event-suppression configurations can depend on their respective parameters. As an illustrative example, the receiver-based event-suppression configuration can identify a set of event types, a severity threshold, a geographic region, and an event-frequency threshold. If the event is associated a first event type, the event-throttling application can apply the receiver-based event-suppression configuration to the event to determine whether the set of event types includes the first event type. If the event is associated a severity metric, the event-throttling application can apply the receiver-based event-suppression configuration to the event to determine whether the severity metric exceeds the severity threshold.

If the transmitted event indicates that subject was located at a particular location during the occurrence of an event, the event-throttling application can also apply the receiver-based event-suppression configuration to the event to determine whether the particular location is within the geographic region. If the event is associated a frequency metric, the event-throttling application can apply the receiver-based event-suppression configuration to the event to determine whether the frequency metric exceeds the event-frequency threshold.

At step 708, the event-throttling application can suppress display or presentation of the event to the receiver device in response to determining that the event should be suppressed. For example, if the severity metric falls below the severity threshold, the event-throttling application determines that the event should be suppressed from being presented or displayed on the receiver device. In another example, if a time metric is outside the time window specified in the receiver-based event-suppression configuration, the event-throttling application can also suppress the event from being presented or displayed on the receiver device.

To suppress the transmission of the event, the event-throttling application can delete the event. In some instances, the event-throttling application can log the event as suppressed, such that additional events associated with the same event type can be used together to determine another parameter (e.g., frequency metric) for determining whether the event should be presented or displayed on to the receiver device later (e.g., comparing the frequency metric to an event-frequency threshold).

Additionally or alternatively, the event-throttling application does not suppress but facilitates presentation of events that are outside the scope of the receiver-based event-suppression configuration. For example, if the event is associated with a second type and the event-throttling application determines that the set of event types does not include the second event type, the event-throttling application can permit and facilitate display of the event on a graphical user interface of the receiver device. Process 700 terminates thereafter.

### II. Example Systems

FIG. 8 illustrates a computing system architecture 800, including various components in electrical communication with each other, in accordance with some embodiments. The example computing system architecture 800 illustrated in FIG. 8 includes a computing device 802, which has various components in electrical communication with each other using a connection 806, such as a bus, in accordance with some implementations. The example computing system architecture 800 includes a processing unit 804 that is in electrical communication with various system components, using the connection 806, and including the system memory 814. In some embodiments, the system memory 814 includes read-only memory (ROM), random-access memory (RAM), and other such memory technologies including, but not limited to, those described herein. In some embodiments, the example computing system architecture 800 includes a cache 808 of high-speed memory connected directly with, in close proximity to, or integrated as part of the processor 804. The system architecture 800 can copy data from the memory 814 and/or the storage device 810 to the cache 808 for quick access by the processor 804. In this way, the cache 808 can provide a performance boost that decreases or eliminates processor delays in the processor 804 due to waiting for data. Using modules, methods and services such as those described herein, the processor 804 can be configured to perform various actions. In some embodiments, the cache 808 may include multiple types of cache including, for example, level one (L1) and level two (L2) cache. The memory 814 may be referred to herein as system memory or computer system memory. The memory 814 may include, at various times, elements of an operating system, one or more applications, data associated with the operating system or the one or more applications, or other such data associated with the computing device 802.

Other system memory 814 can be available for use as well. The memory 814 can include multiple different types of memory with different performance characteristics. The processor 804 can include any general purpose processor and one or more hardware or software services, such as service 812 stored in storage device 810, configured to control the processor 804 as well as a special-purpose processor where software instructions are incorporated into the actual processor design. The processor 804 can be a completely self-contained computing system, containing multiple cores or processors, connectors (e.g., buses), memory, memory controllers, caches, etc. In some embodiments, such a self-contained computing system with multiple cores is symmetric. In some embodiments, such a self-contained computing system with multiple cores is asymmetric. In some embodiments, the processor 804 can be a microprocessor, a microcontroller, a digital signal processor (“DSP”), or a combination of these and/or other types of processors. In some embodiments, the processor 804 can include multiple elements such as a core, one or more registers, and one or more processing units such as an arithmetic logic unit (ALU), a floating point unit (FPU), a graphics processing unit (GPU), a physics processing unit (PPU), a digital system processing (DSP) unit, or combinations of these and/or other such processing units.

To enable user interaction with the computing system architecture 800, an input device 816 can represent any number of input mechanisms, such as a microphone for speech, a touch-sensitive screen for gesture or graphical input, keyboard, mouse, motion input, pen, and other such input devices. An output device 818 can also be one or more of a number of output mechanisms known to those of skill in the art including, but not limited to, monitors, speakers, printers, haptic devices, and other such output devices. In some instances, multimodal systems can enable a user to provide multiple types of input to communicate with the computing system architecture 800. In some embodiments, the input device 816 and/or the output device 818 can be coupled to the computing device 802 using a remote connection device such as, for example, a communication interface such as the network interface 820 described herein. In such embodiments, the communication interface can govern and manage the input and output received from the attached input device 816 and/or output device 818. As may be contemplated, there is no restriction on operating on any particular hardware arrangement and accordingly the basic features here may easily be substituted for other hardware, software, or firmware arrangements as they are developed.

In some embodiments, the storage device 810 can be described as non-volatile storage or non-volatile memory. Such non-volatile memory or non-volatile storage can be a hard disk or other types of computer readable media which can store data that are accessible by a computer, such as magnetic cassettes, flash memory cards, solid state memory devices, digital versatile disks, cartridges, RAM, ROM, and hybrids thereof.

As described above, the storage device 810 can include hardware and/or software services such as service 812 that can control or configure the processor 804 to perform one or more functions including, but not limited to, the methods, processes, functions, systems, and services described herein in various embodiments. In some embodiments, the hardware or software services can be implemented as modules. As illustrated in example computing system architecture 800, the storage device 810 can be connected to other parts of the computing device 802 using the system connection 806. In some embodiments, a hardware service or hardware module such as service 812, that performs a function can include a software component stored in a non-transitory computer-readable medium that, in connection with the necessary hardware components, such as the processor 804, connection 806, cache 808, storage device 810, memory 814, input device 816, output device 818, and so forth, can carry out the functions such as those described herein.

The disclosed systems and service of a event-processing application (e.g., the event-processing application 402 described herein at least in connection with FIG. 4) can be performed using a computing system such as the example computing system illustrated in FIG. 8, using one or more components of the example computing system architecture 800. An example computing system can include a processor (e.g., a central processing unit), memory, non-volatile memory, and an interface device. The memory may store data and/or and one or more code sets, software, scripts, etc. The components of the computer system can be coupled together via a bus or through some other known or convenient device.

In some embodiments, the processor can be configured to carry out some or all of methods and systems for transmitting or suppressing events associated with the event-processing application (e.g., the event-processing application 402 described herein at least in connection with FIG. 4) described herein by, for example, executing code using a processor such as processor 804 wherein the code is stored in memory such as memory 814 as described herein. One or more of a user device, a provider server or system, a database system, or other such devices, services, or systems may include some or all of the components of the computing system such as the example computing system illustrated in FIG. 8, using one or more components of the example computing system architecture 800 illustrated herein. As may be contemplated, variations on such systems can be considered as within the scope of the present disclosure.

This disclosure contemplates the computer system taking any suitable physical form. As example and not by way of limitation, the computer system can be an embedded computer system, a system-on-chip (SOC), a single-board computer system (SBC) (such as, for example, a computer-on-module (COM) or system-on-module (SOM)), a desktop computer system, a laptop or notebook computer system, a tablet computer system, a wearable computer system or interface, an interactive kiosk, a mainframe, a mesh of computer systems, a mobile telephone, a personal digital assistant (PDA), a server, or a combination of two or more of these. Where appropriate, the computer system may include one or more computer systems; be unitary or distributed; span multiple locations; span multiple machines; and/or reside in a cloud computing system which may include one or more cloud components in one or more networks as described herein in association with the computing resources provider 828. Where appropriate, one or more computer systems may perform without substantial spatial or temporal limitation one or more steps of one or more methods described or illustrated herein. As an example and not by way of limitation, one or more computer systems may perform in real time or in batch mode one or more steps of one or more methods described or illustrated herein. One or more computer systems may perform at different times or at different locations one or more steps of one or more methods described or illustrated herein, where appropriate.

The processor 804 can be a conventional microprocessor such as an Intel® microprocessor, an AMD® microprocessor, a Motorola® microprocessor, or other such microprocessors. One of skill in the relevant art will recognize that the terms “machine-readable (storage) medium” or “computer-readable (storage) medium” include any type of device that is accessible by the processor.

The memory 814 can be coupled to the processor 804 by, for example, a connector such as connector 806, or a bus. As used herein, a connector or bus such as connector 806 is a communications system that transfers data between components within the computing device 802 and may, in some embodiments, be used to transfer data between computing devices. The connector 806 can be a data bus, a memory bus, a system bus, or other such data transfer mechanism. Examples of such connectors include, but are not limited to, an industry standard architecture (ISA” bus, an extended ISA (EISA) bus, a parallel AT attachment (PATA” bus (e.g., an integrated drive electronics (IDE) or an extended IDE (EIDE) bus), or the various types of parallel component interconnect (PCI) buses (e.g., PCI, PCIe, PCI-104, etc.).

The memory 814 can include RAM including, but not limited to, dynamic RAM (DRAM), static RAM (SRAM), synchronous dynamic RAM (SDRAM), non-volatile random access memory (NVRAM), and other types of RAM. The DRAM may include error-correcting code (EEC). The memory can also include ROM including, but not limited to, programmable ROM (PROM), erasable and programmable ROM (EPROM), electronically erasable and programmable ROM (EEPROM), Flash Memory, masked ROM (MROM), and other types or ROM. The memory 814 can also include magnetic or optical data storage media including read-only (e.g., CD ROM and DVD ROM) or otherwise (e.g., CD or DVD). The memory can be local, remote, or distributed.

As described above, the connector 806 (or bus) can also couple the processor 804 to the storage device 810, which may include non-volatile memory or storage and which may also include a drive unit. In some embodiments, the non-volatile memory or storage is a magnetic floppy or hard disk, a magnetic-optical disk, an optical disk, a ROM (e.g., a CD-ROM, DVD-ROM, EPROM, or EEPROM), a magnetic or optical card, or another form of storage for data. Some of this data may be written, by a direct memory access process, into memory during execution of software in a computer system. The non-volatile memory or storage can be local, remote, or distributed. In some embodiments, the non-volatile memory or storage is optional. As may be contemplated, a computing system can be created with all applicable data available in memory. A typical computer system will usually include at least one processor, memory, and a device (e.g., a bus) coupling the memory to the processor.

Software and/or data associated with software can be stored in the non-volatile memory and/or the drive unit. In some embodiments (e.g., for large programs) it may not be possible to store the entire program and/or data in the memory at any one time. In such embodiments, the program and/or data can be moved in and out of memory from, for example, an additional storage device such as storage device 810. Nevertheless, it should be understood that for software to run, if necessary, it is moved to a computer readable location appropriate for processing, and for illustrative purposes, that location is referred to as the memory herein. Even when software is moved to the memory for execution, the processor can make use of hardware registers to store values associated with the software, and local cache that, ideally, serves to speed up execution. As used herein, a software program is assumed to be stored at any known or convenient location (from non-volatile storage to hardware registers), when the software program is referred to as “implemented in a computer-readable medium.” A processor is considered to be “configured to execute a program” when at least one value associated with the program is stored in a register readable by the processor.

The connection 806 can also couple the processor 804 to a network interface device such as the network interface 820. The interface can include one or more of a modem or other such network interfaces including, but not limited to those described herein. It will be appreciated that the network interface 820 may be considered to be part of the computing device 802 or may be separate from the computing device 802. The network interface 820 can include one or more of an analog modem, Integrated Services Digital Network (ISDN) modem, cable modem, token ring interface, satellite transmission interface, or other interfaces for coupling a computer system to other computer systems. In some embodiments, the network interface 820 can include one or more input and/or output (I/O) devices. The I/O devices can include, by way of example but not limitation, input devices such as input device 816 and/or output devices such as output device 818. For example, the network interface 820 may include a keyboard, a mouse, a printer, a scanner, a display device, and other such components. Other examples of input devices and output devices are described herein. In some embodiments, a communication interface device can be implemented as a complete and separate computing device.

In operation, the computer system can be controlled by operating system software that includes a file management system, such as a disk operating system. One example of operating system software with associated file management system software is the family of Windows® operating systems and their associated file management systems. Another example of operating system software with its associated file management system software is the Linux™ operating system and its associated file management system including, but not limited to, the various types and implementations of the Linux® operating system and their associated file management systems. The file management system can be stored in the non-volatile memory and/or drive unit and can cause the processor to execute the various acts required by the operating system to input and output data and to store data in the memory, including storing files on the non-volatile memory and/or drive unit. As may be contemplated, other types of operating systems such as, for example, MacOS®, other types of UNIX® operating systems (e.g., BSD™ and descendants, Xenix™, SunOS™, HP-UX®, etc.), mobile operating systems (e.g., iOS® and variants, Chrome®, Ubuntu Touch®, watchOS®, Windows 10 Mobile®, the Blackberry® OS, etc.), and real-time operating systems (e.g., VxWorks®, QNX®, eCos®, RTLinux®, etc.) may be considered as within the scope of the present disclosure. As may be contemplated, the names of operating systems, mobile operating systems, real-time operating systems, languages, and devices, listed herein may be registered trademarks, service marks, or designs of various associated entities.

In some embodiments, the computing device 802 can be connected to one or more additional computing devices such as computing device 824 via a network 822 using a connection such as the network interface 820. In such embodiments, the computing device 824 may execute one or more services 826 to perform one or more functions under the control of, or on behalf of, programs and/or services operating on computing device 802. In some embodiments, a computing device such as computing device 824 may include one or more of the types of components as described in connection with computing device 802 including, but not limited to, a processor such as processor 804, a connection such as connection 806, a cache such as cache 808, a storage device such as storage device 810, memory such as memory 814, an input device such as input device 816, and an output device such as output device 818. In such embodiments, the computing device 824 can carry out the functions such as those described herein in connection with computing device 802. In some embodiments, the computing device 802 can be connected to a plurality of computing devices such as computing device 824, each of which may also be connected to a plurality of computing devices such as computing device 824. Such an embodiment may be referred to herein as a distributed computing environment.

The network 822 can be any network including an internet, an intranet, an extranet, a cellular network, a Wi-Fi network, a local area network (LAN), a wide area network (WAN), a satellite network, a Bluetooth® network, a virtual private network (VPN), a public switched telephone network, an infrared (IR) network, an internet of things (IoT network) or any other such network or combination of networks. Communications via the network 822 can be wired connections, wireless connections, or combinations thereof. Communications via the network 822 can be made via a variety of communications protocols including, but not limited to, Transmission Control Protocol/Internet Protocol (TCP/IP), User Datagram Protocol (UDP), protocols in various layers of the Open System Interconnection (OSI) model, File Transfer Protocol (FTP), Universal Plug and Play (UPnP), Network File System (NFS), Server Message Block (SMB), Common Internet File System (CIFS), CANBus, and other such communications protocols.

Communications over the network 822, within the computing device 802, within the computing device 824, or within the computing resources provider 828 can include information, which also may be referred to herein as content. The information may include text, graphics, audio, video, haptics, and/or any other information that can be provided to a user of the computing device such as the computing device 802. In some embodiments, the information can be delivered using a transfer protocol such as Hypertext Markup Language (HTML), Extensible Markup Language (XML), JavaScript®, Cascading Style Sheets (CSS), JavaScript® Object Notation (JSON), Protobuf, Msgpack, Thrift, and other such protocols and/or structured languages. The information may first be processed by the computing device 802 and presented to a user of the computing device 802 using forms that are perceptible via sight, sound, smell, taste, touch, or other such mechanisms. In some embodiments, communications over the network 822 can be received and/or processed by a computing device configured as a server. Such communications can be sent and received using PHP: Hypertext Preprocessor (“PHP”), Python™, Ruby, Perl® and variants, Java®, HTML, XML, or another such server-side processing language.

In some embodiments, the computing device 802 and/or the computing device 824 can be connected to a computing resources provider 828 via the network 822 using a network interface such as those described herein (e.g. network interface 820). In such embodiments, one or more systems (e.g., service 830 and service 832) hosted within the computing resources provider 828 (also referred to herein as within “a computing resources provider environment”) may execute one or more services to perform one or more functions under the control of, or on behalf of, programs and/or services operating on computing device 802 and/or computing device 824. Systems such as service 830 and service 832 may include one or more computing devices such as those described herein to execute computer code to perform the one or more functions under the control of, or on behalf of, programs and/or services operating on computing device 802 and/or computing device 824.

For example, the computing resources provider 828 may provide a service, operating on service 830 to store data for the computing device 802 when, for example, the amount of data that the computing device 802 exceeds the capacity of storage device 810. In another example, the computing resources provider 828 may provide a service to first instantiate a virtual machine (VM) on service 832, use that VM to access the data stored on service 832, perform one or more operations on that data, and provide a result of those one or more operations to the computing device 802. Such operations (e.g., data storage and VM instantiation) may be referred to herein as operating “in the cloud,” “within a cloud computing environment,” or “within a hosted virtual machine environment,” and the computing resources provider 828 may also be referred to herein as “the cloud.” Examples of such computing resources providers include, but are not limited to Amazon® Web Services (AWS®), Microsoft's Azure®, IBM Cloud®, Google Cloud®, Oracle Cloud® etc.

Services provided by a computing resources provider 828 include, but are not limited to, data analytics, data storage, archival storage, big data storage, virtual computing (including various scalable VM architectures), blockchain services, containers (e.g., application encapsulation), database services, development environments (including sandbox development environments), e-commerce solutions, game services, media and content management services, security services, server-less hosting, virtual reality (VR) systems, and augmented reality (AR) systems. Various techniques to facilitate such services include, but are not be limited to, virtual machines, virtual storage, database services, system schedulers (e.g., hypervisors), resource management systems, various types of short-term, mid-term, long-term, and archival storage devices, etc.

As may be contemplated, the systems such as service 830 and service 832 may implement versions of various services (e.g., the service 812 or the service 826) on behalf of, or under the control of, computing device 802 and/or computing device 824. Such implemented versions of various services may involve one or more virtualization techniques so that, for example, it may appear to a user of computing device 802 that the service 812 is executing on the computing device 802 when the service is executing on, for example, service 830. As may also be contemplated, the various services operating within the computing resources provider 828 environment may be distributed among various systems within the environment as well as partially distributed onto computing device 824 and/or computing device 802.

Client devices, user devices, computer resources provider devices, network devices, and other devices can be computing systems that include one or more integrated circuits, input devices, output devices, data storage devices, and/or network interfaces, among other things. The integrated circuits can include, for example, one or more processors, volatile memory, and/or non-volatile memory, among other things such as those described herein. The input devices can include, for example, a keyboard, a mouse, a key pad, a touch interface, a microphone, a camera, VR/AR input device (e.g., AR glasses), and/or other types of input devices including, but not limited to, those described herein. The output devices can include, for example, a display screen, a speaker, a haptic feedback system, a printer, and/or other types of output devices including, but not limited to, those described herein. A data storage device, such as a hard drive or flash memory, can enable the computing device to temporarily or permanently store data. A network interface, such as a wireless or wired interface, can enable the computing device to communicate with a network. Examples of computing devices (e.g., the computing device 802) include, but is not limited to, desktop computers, laptop computers, server computers, hand-held computers, tablets, smart phones, personal digital assistants, digital home assistants, wearable devices, smart devices, and combinations of these and/or other such computing devices as well as machines and apparatuses in which a computing device has been incorporated and/or virtually implemented.

The techniques described herein may also be implemented in electronic hardware, computer software, firmware, or any combination thereof. Such techniques may be implemented in any of a variety of devices such as general purpose computers, wireless communication device handsets, or integrated circuit devices having multiple uses including application in wireless communication device handsets and other devices. Any features described as modules or components may be implemented together in an integrated logic device or separately as discrete but interoperable logic devices. If implemented in software, the techniques may be realized at least in part by a computer-readable data storage medium comprising program code including instructions that, when executed, performs one or more of the methods described above. The computer-readable data storage medium may form part of a computer program product, which may include packaging materials. The computer-readable medium may comprise memory or data storage media, such as that described herein. The techniques additionally, or alternatively, may be realized at least in part by a computer-readable communication medium that carries or communicates program code in the form of instructions or data structures and that can be accessed, read, and/or executed by a computer, such as propagated signals or waves.

The program code may be executed by a processor, which may include one or more processors, such as one or more digital signal processors (DSPs), general purpose microprocessors, an application specific integrated circuits (ASICs), field programmable logic arrays (FPGAs), or other equivalent integrated or discrete logic circuitry. Such a processor may be configured to perform any of the techniques described in this disclosure. A general purpose processor may be a microprocessor; but in the alternative, the processor may be any conventional processor, controller, microcontroller, or state machine. A processor may also be implemented as a combination of computing devices (e.g., a combination of a DSP and a microprocessor), a plurality of microprocessors, one or more microprocessors in conjunction with a DSP core, or any other such configuration. Accordingly, the term “processor,” as used herein may refer to any of the foregoing structure, any combination of the foregoing structure, or any other structure or apparatus suitable for implementation of the techniques described herein. In addition, in some aspects, the functionality described herein may be provided within dedicated software modules or hardware modules configured for implementing a suspended database update system.

As used herein, the term “machine-readable media” and equivalent terms “machine-readable storage media,” “computer-readable media,” and “computer-readable storage media” refer to media that includes, but is not limited to, portable or non-portable storage devices, optical storage devices, removable or non-removable storage devices, and various other mediums capable of storing, containing, or carrying instruction(s) and/or data. A computer-readable medium may include a non-transitory medium in which data can be stored and that does not include carrier waves and/or transitory electronic signals propagating wirelessly or over wired connections. Examples of a non-transitory medium may include, but are not limited to, a magnetic disk or tape, optical storage media such as compact disk (CD) or digital versatile disk (DVD), solid state drives (SSD), flash memory, memory or memory devices.

A machine-readable medium or machine-readable storage medium may have stored thereon code and/or machine-executable instructions that may represent a procedure, a function, a subprogram, a program, a routine, a subroutine, a module, a software package, a class, or any combination of instructions, data structures, or program statements. A code segment may be coupled to another code segment or a hardware circuit by passing and/or receiving information, data, arguments, parameters, or memory contents. Information, arguments, parameters, data, etc. may be passed, forwarded, or transmitted via any suitable means including memory sharing, message passing, token passing, network transmission, or the like. Further examples of machine-readable storage media, machine-readable media, or computer-readable (storage) media include but are not limited to recordable type media such as volatile and non-volatile memory devices, floppy and other removable disks, hard disk drives, optical disks (e.g., CDs, DVDs, etc.), among others, and transmission type media such as digital and analog communication links.

As may be contemplated, while examples herein may illustrate or refer to a machine-readable medium or machine-readable storage medium as a single medium, the term “machine-readable medium” and “machine-readable storage medium” should be taken to include a single medium or multiple media (e.g., a centralized or distributed database, and/or associated caches and servers) that store the one or more sets of instructions. The term “machine-readable medium” and “machine-readable storage medium” shall also be taken to include any medium that is capable of storing, encoding, or carrying a set of instructions for execution by the system and that cause the system to perform any one or more of the methodologies or modules of disclosed herein.

Some portions of the detailed description herein may be presented in terms of algorithms and symbolic representations of operations on data bits within a computer memory. These algorithmic descriptions and representations are the means used by those skilled in the data processing arts to most effectively convey the substance of their work to others skilled in the art. An algorithm is here, and generally, conceived to be a self-consistent sequence of operations leading to a desired result. The operations are those requiring physical manipulations of physical quantities. Usually, though not necessarily, these quantities take the form of electrical or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind, however, that all of these and similar terms are to be associated with the appropriate physical quantities and are merely convenient labels applied to these quantities. Unless specifically stated otherwise as apparent from the following discussion, it is appreciated that throughout the description, discussions utilizing terms such as “processing” or “computing” or “calculating” or “determining” or “displaying” or “generating” or the like, refer to the action and processes of a computer system, or similar electronic computing device, that manipulates and transforms data represented as physical (electronic) quantities within registers and memories of the computer system into other data similarly represented as physical quantities within the computer system memories or registers or other such information storage, transmission or display devices.

It is also noted that individual implementations may be described as a process which is depicted as a flowchart, a flow diagram, a data flow diagram, a structure diagram, or a block diagram (e.g., the example process 500 of FIG. 5, the example process 700 of FIG. 7). Although a flowchart, a flow diagram, a data flow diagram, a structure diagram, or a block diagram may describe the operations as a sequential process, many of the operations can be performed in parallel or concurrently. In addition, the order of the operations may be re-arranged. A process illustrated in a figure is terminated when its operations are completed, but could have additional steps not included in the figure. A process may correspond to a method, a function, a procedure, a subroutine, a subprogram, etc. When a process corresponds to a function, its termination can correspond to a return of the function to the calling function or the main function.

In some embodiments, one or more implementations of an algorithm such as those described herein may be implemented using a machine learning or artificial intelligence algorithm. Such a machine learning or artificial intelligence algorithm may be trained using supervised, unsupervised, reinforcement, or other such training techniques. For example, a set of data may be analyzed using one of a variety of machine learning algorithms to identify correlations between different elements of the set of data without supervision and feedback (e.g., an unsupervised training technique). A machine learning data analysis algorithm may also be trained using sample or live data to identify potential correlations. Such algorithms may include k-means clustering algorithms, fuzzy c-means (FCM) algorithms, expectation-maximization (EM) algorithms, hierarchical clustering algorithms, density-based spatial clustering of applications with noise (DBSCAN) algorithms, and the like. Other examples of machine learning or artificial intelligence algorithms include, but are not limited to, genetic algorithms, backpropagation, reinforcement learning, decision trees, liner classification, artificial neural networks, anomaly detection, and such. More generally, machine learning or artificial intelligence methods may include regression analysis, dimensionality reduction, metalearning, reinforcement learning, deep learning, and other such algorithms and/or methods. As may be contemplated, the terms “machine learning” and “artificial intelligence” are frequently used interchangeably due to the degree of overlap between these fields and many of the disclosed techniques and algorithms have similar approaches.

As an example of a supervised training technique, a set of data can be selected for training of the machine learning model to facilitate identification of correlations between members of the set of data. The machine learning model may be evaluated to determine, based on the sample inputs supplied to the machine learning model, whether the machine learning model is producing accurate correlations between members of the set of data. Based on this evaluation, the machine learning model may be modified to increase the likelihood of the machine learning model identifying the desired correlations. The machine learning model may further be dynamically trained by soliciting feedback from users of a system as to the efficacy of correlations provided by the machine learning algorithm or artificial intelligence algorithm (i.e., the supervision). The machine learning algorithm or artificial intelligence may use this feedback to improve the algorithm for generating correlations (e.g., the feedback may be used to further train the machine learning algorithm or artificial intelligence to provide more accurate correlations).

The various examples of flowcharts, flow diagrams, data flow diagrams, structure diagrams, or block diagrams discussed herein may further be implemented by hardware, software, firmware, middleware, microcode, hardware description languages, or any combination thereof. When implemented in software, firmware, middleware or microcode, the program code or code segments to perform the necessary tasks (e.g., a computer-program product) may be stored in a computer-readable or machine-readable storage medium (e.g., a medium for storing program code or code segments) such as those described herein. A processor(s), implemented in an integrated circuit, may perform the necessary tasks.

The various illustrative logical blocks, modules, circuits, and algorithm steps described in connection with the implementations disclosed herein may be implemented as electronic hardware, computer software, firmware, or combinations thereof. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of the present disclosure.

It should be noted, however, that the algorithms and displays presented herein are not inherently related to any particular computer or other apparatus. Various general purpose systems may be used with programs in accordance with the teachings herein, or it may prove convenient to construct more specialized apparatus to perform the methods of some examples. The required structure for a variety of these systems will appear from the description below. In addition, the techniques are not described with reference to any particular programming language, and various examples may thus be implemented using a variety of programming languages.

In various implementations, the system operates as a standalone device or may be connected (e.g., networked) to other systems. In a networked deployment, the system may operate in the capacity of a server or a client system in a client-server network environment, or as a peer system in a peer-to-peer (or distributed) network environment.

The system may be a server computer, a client computer, a personal computer (PC), a tablet PC (e.g., an iPad®, a Microsoft Surface®, a Chromebook®, etc.), a laptop computer, a set-top box (STB), a personal digital assistants (PDA), a mobile device (e.g., a cellular telephone, an iPhone®, and Android® device, a Blackberry®, etc.), a wearable device, an embedded computer system, an electronic book reader, a processor, a telephone, a web appliance, a network router, switch or bridge, or any system capable of executing a set of instructions (sequential or otherwise) that specify actions to be taken by that system. The system may also be a virtual system such as a virtual version of one of the aforementioned devices that may be hosted on another computer device such as the computer device 802.

In general, the routines executed to implement the implementations of the disclosure, may be implemented as part of an operating system or a specific application, component, program, object, module or sequence of instructions referred to as “computer programs.” The computer programs typically comprise one or more instructions set at various times in various memory and storage devices in a computer, and that, when read and executed by one or more processing units or processors in a computer, cause the computer to perform operations to execute elements involving the various aspects of the disclosure.

Moreover, while examples have been described in the context of fully functioning computers and computer systems, those skilled in the art will appreciate that the various examples are capable of being distributed as a program object in a variety of forms, and that the disclosure applies equally regardless of the particular type of machine or computer-readable media used to actually effect the distribution.

In some circumstances, operation of a memory device, such as a change in state from a binary one to a binary zero or vice-versa, for example, may comprise a transformation, such as a physical transformation. With particular types of memory devices, such a physical transformation may comprise a physical transformation of an article to a different state or thing. For example, but without limitation, for some types of memory devices, a change in state may involve an accumulation and storage of charge or a release of stored charge. Likewise, in other memory devices, a change of state may comprise a physical change or transformation in magnetic orientation or a physical change or transformation in molecular structure, such as from crystalline to amorphous or vice versa. The foregoing is not intended to be an exhaustive list of all examples in which a change in state for a binary one to a binary zero or vice-versa in a memory device may comprise a transformation, such as a physical transformation. Rather, the foregoing is intended as illustrative examples.

A storage medium typically may be non-transitory or comprise a non-transitory device. In this context, a non-transitory storage medium may include a device that is tangible, meaning that the device has a concrete physical form, although the device may change its physical state. Thus, for example, non-transitory refers to a device remaining tangible despite this change in state.

The above description and drawings are illustrative and are not to be construed as limiting or restricting the subject matter to the precise forms disclosed. Persons skilled in the relevant art can appreciate that many modifications and variations are possible in light of the above disclosure and may be made thereto without departing from the broader scope of the embodiments as set forth herein. Numerous specific details are described to provide a thorough understanding of the disclosure. However, in certain instances, well-known or conventional details are not described in order to avoid obscuring the description.

As used herein, the terms “connected,” “coupled,” or any variant thereof when applying to modules of a system, means any connection or coupling, either direct or indirect, between two or more elements; the coupling of connection between the elements can be physical, logical, or any combination thereof. Additionally, the words “herein,” “above,” “below,” and words of similar import, when used in this application, shall refer to this application as a whole and not to any particular portions of this application. Where the context permits, words in the above Detailed Description using the singular or plural number may also include the plural or singular number respectively. The word “or,” in reference to a list of two or more items, covers all of the following interpretations of the word: any of the items in the list, all of the items in the list, or any combination of the items in the list.

As used herein, the terms “a” and “an” and “the” and other such singular referents are to be construed to include both the singular and the plural, unless otherwise indicated herein or clearly contradicted by context.

As used herein, the terms “comprising,” “having,” “including,” and “containing” are to be construed as open-ended (e.g., “including” is to be construed as “including, but not limited to”), unless otherwise indicated or clearly contradicted by context.

As used herein, the recitation of ranges of values is intended to serve as a shorthand method of referring individually to each separate value falling within the range, unless otherwise indicated or clearly contradicted by context. Accordingly, each separate value of the range is incorporated into the specification as if it were individually recited herein.

As used herein, use of the terms “set” (e.g., “a set of items”) and “subset” (e.g., “a subset of the set of items”) is to be construed as a nonempty collection including one or more members unless otherwise indicated or clearly contradicted by context. Furthermore, unless otherwise indicated or clearly contradicted by context, the term “subset” of a corresponding set does not necessarily denote a proper subset of the corresponding set but that the subset and the set may include the same elements (i.e., the set and the subset may be the same).

As used herein, use of conjunctive language such as “at least one of A, B, and C” is to be construed as indicating one or more of A, B, and C (e.g., any one of the following nonempty subsets of the set {A, B, C}, namely: {A}, {B}, {C}, {A, B}, {A, C}, {B, C}, or {A, B, C}) unless otherwise indicated or clearly contradicted by context. Accordingly, conjunctive language such as “as least one of A, B, and C” does not imply a requirement for at least one of A, at least one of B, and at least one of C.

As used herein, the use of examples or exemplary language (e.g., “such as” or “as an example”) is intended to more clearly illustrate embodiments and does not impose a limitation on the scope unless otherwise claimed. Such language in the specification should not be construed as indicating any non-claimed element is required for the practice of the embodiments described and claimed in the present disclosure.

As used herein, where components are described as being “configured to” perform certain operations, such configuration can be accomplished, for example, by designing electronic circuits or other hardware to perform the operation, by programming programmable electronic circuits (e.g., microprocessors, or other suitable electronic circuits) to perform the operation, or any combination thereof.

Those of skill in the art will appreciate that the disclosed subject matter may be embodied in other forms and manners not shown below. It is understood that the use of relational terms, if any, such as first, second, top and bottom, and the like are used solely for distinguishing one entity or action from another, without necessarily requiring or implying any such actual relationship or order between such entities or actions.

While processes or blocks are presented in a given order, alternative implementations may perform routines having steps, or employ systems having blocks, in a different order, and some processes or blocks may be deleted, moved, added, subdivided, substituted, combined, and/or modified to provide alternative or sub combinations. Each of these processes or blocks may be implemented in a variety of different ways. Also, while processes or blocks are at times shown as being performed in series, these processes or blocks may instead be performed in parallel, or may be performed at different times. Further any specific numbers noted herein are only examples: alternative implementations may employ differing values or ranges.

The teachings of the disclosure provided herein can be applied to other systems, not necessarily the system described above. The elements and acts of the various examples described above can be combined to provide further examples.

Any patents and applications and other references noted above, including any that may be listed in accompanying filing papers, are incorporated herein by reference. Aspects of the disclosure can be modified, if necessary, to employ the systems, functions, and concepts of the various references described above to provide yet further examples of the disclosure.

These and other changes can be made to the disclosure in light of the above Detailed Description. While the above description describes certain examples, and describes the best mode contemplated, no matter how detailed the above appears in text, the teachings can be practiced in many ways. Details of the system may vary considerably in its implementation details, while still being encompassed by the subject matter disclosed herein. As noted above, particular terminology used when describing certain features or aspects of the disclosure should not be taken to imply that the terminology is being redefined herein to be restricted to any specific characteristics, features, or aspects of the disclosure with which that terminology is associated. In general, the terms used in the following claims should not be construed to limit the disclosure to the specific implementations disclosed in the specification, unless the above Detailed Description section explicitly defines such terms. Accordingly, the actual scope of the disclosure encompasses not only the disclosed implementations, but also all equivalent ways of practicing or implementing the disclosure under the claims.

While certain aspects of the disclosure are presented below in certain claim forms, the inventors contemplate the various aspects of the disclosure in any number of claim forms. Any claims intended to be treated under 45 U.S.C. § 112(f) will begin with the words “means for”. Accordingly, the applicant reserves the right to add additional claims after filing the application to pursue such additional claim forms for other aspects of the disclosure.

The terms used in this specification generally have their ordinary meanings in the art, within the context of the disclosure, and in the specific context where each term is used. Certain terms that are used to describe the disclosure are discussed above, or elsewhere in the specification, to provide additional guidance to the practitioner regarding the description of the disclosure. For convenience, certain terms may be highlighted, for example using capitalization, italics, and/or quotation marks. The use of highlighting has no influence on the scope and meaning of a term; the scope and meaning of a term is the same, in the same context, whether or not it is highlighted. It will be appreciated that same element can be described in more than one way.

Consequently, alternative language and synonyms may be used for any one or more of the terms discussed herein, nor is any special significance to be placed upon whether or not a term is elaborated or discussed herein. Synonyms for certain terms are provided. A recital of one or more synonyms does not exclude the use of other synonyms. The use of examples anywhere in this specification including examples of any terms discussed herein is illustrative only, and is not intended to further limit the scope and meaning of the disclosure or of any exemplified term. Likewise, the disclosure is not limited to various examples given in this specification.

Without intent to further limit the scope of the disclosure, examples of instruments, apparatus, methods and their related results according to the examples of the present disclosure are given below. Note that titles or subtitles may be used in the examples for convenience of a reader, which in no way should limit the scope of the disclosure. Unless otherwise defined, all technical and scientific terms used herein have the same meaning as commonly understood by one of ordinary skill in the art to which this disclosure pertains. In the case of conflict, the present document, including definitions will control.

Some portions of this description describe examples in terms of algorithms and symbolic representations of operations on information. These algorithmic descriptions and representations are commonly used by those skilled in the data processing arts to convey the substance of their work effectively to others skilled in the art. These operations, while described functionally, computationally, or logically, are understood to be implemented by computer programs or equivalent electrical circuits, microcode, or the like. Furthermore, it has also proven convenient at times, to refer to these arrangements of operations as modules, without loss of generality. The described operations and their associated modules may be embodied in software, firmware, hardware, or any combinations thereof.

Any of the steps, operations, or processes described herein may be performed or implemented with one or more hardware or software modules, alone or in combination with other devices. In some examples, a software module is implemented with a computer program object comprising a computer-readable medium containing computer program code, which can be executed by a computer processor for performing any or all of the steps, operations, or processes described.

Examples may also relate to an apparatus for performing the operations herein. This apparatus may be specially constructed for the required purposes, and/or it may comprise a general-purpose computing device selectively activated or reconfigured by a computer program stored in the computer. Such a computer program may be stored in a non-transitory, tangible computer readable storage medium, or any type of media suitable for storing electronic instructions, which may be coupled to a computer system bus. Furthermore, any computing systems referred to in the specification may include a single processor or may be architectures employing multiple processor designs for increased computing capability.

Examples may also relate to an object that is produced by a computing process described herein. Such an object may comprise information resulting from a computing process, where the information is stored on a non-transitory, tangible computer readable storage medium and may include any implementation of a computer program object or other data combination described herein.

The language used in the specification has been principally selected for readability and instructional purposes, and it may not have been selected to delineate or circumscribe the subject matter. It is therefore intended that the scope of this disclosure be limited not by this detailed description, but rather by any claims that issue on an application based hereon. Accordingly, the disclosure of the examples is intended to be illustrative, but not limiting, of the scope of the subject matter, which is set forth in the following claims.

Specific details were given in the preceding description to provide a thorough understanding of various implementations of systems and components for a contextual connection system. It will be understood by one of ordinary skill in the art, however, that the implementations described above may be practiced without these specific details. For example, circuits, systems, networks, processes, and other components may be shown as components in block diagram form in order not to obscure the embodiments in unnecessary detail. In other instances, well-known circuits, processes, algorithms, structures, and techniques may be shown without unnecessary detail in order to avoid obscuring the embodiments.

The foregoing detailed description of the technology has been presented for purposes of illustration and description. It is not intended to be exhaustive or to limit the technology to the precise form disclosed. Many modifications and variations are possible in light of the above teaching. The described embodiments were chosen in order to best explain the principles of the technology, its practical application, and to enable others skilled in the art to utilize the technology in various embodiments and with various modifications as are suited to the particular use contemplated. It is intended that the scope of the technology be defined by the claim.

What is claimed is:

1. A computer-implemented method comprising:
receiving sensor data collected by one or more sensors associated with a sender device;
determining an occurrence of an event associated with a subject of the sender device, wherein the event is determined based on the sensor data, and wherein the event is associated with a first event type;
identifying an event-suppression configuration associated with a receiver device, wherein the event-suppression configuration identifies a set of event types;
applying the event-suppression configuration to the event associated with the subject, wherein applying the event-suppression configuration includes determining whether the set of event types includes the first event type;
determining that the set of event types includes the first event type; and
suppressing transmission of the event to the receiver device.

2. The computer-implemented method of claim 1, wherein the event is associated with a severity metric, the method further comprising:
identifying a second event-suppression configuration associated with a second receiver device, wherein the second event-suppression configuration identifies a severity threshold;
applying the second event-suppression configuration to the event associated with the subject, wherein applying the second event-suppression configuration includes determining whether the severity metric exceeds the severity threshold;
determining that the severity metric does not exceed the severity threshold; and
suppressing transmission of the event to the second receiver device.

3. The computer-implemented method of claim 1, wherein the sensor data indicates that the sender device was located at a particular location during the occurrence of the event, the method further comprising:
identifying a third event-suppression configuration associated with a third receiver device, wherein the third event-suppression configuration identifies a geographic region;
applying the third event-suppression configuration to the event associated with the subject, wherein applying the third event-suppression configuration includes determining whether the particular location is within the geographic region;
determining that the particular location is within the geographic region; and
suppressing transmission of the event to the third receiver device.

4. The computer-implemented method of claim 1, wherein the event is associated with a frequency metric, the method further comprising:
identifying a fourth event-suppression configuration associated with a fourth receiver device, wherein the fourth event-suppression configuration an event-frequency threshold;
applying the fourth event-suppression configuration to the event associated with the subject, wherein applying the fourth event-suppression configuration includes determining whether the frequency metric exceeds the event-frequency threshold;
determining that the frequency metric does not exceed the event-frequency threshold; and
suppressing transmission of the event to the fourth receiver device.

5. The computer-implemented method of claim 1, wherein determining the occurrence of the event includes applying a machine-learning model to the sensor data to determine the occurrence of the event.

6. The computer-implemented method of claim 1, further comprising:
determining an occurrence of a second event associated with the subject, wherein the second event is determined based on the sensor data, and wherein the second event is associated with a second event type;
applying the event-suppression configuration to the second event associated with the subject, wherein applying the event-suppression configuration includes determining whether the set of event types includes the second event type;
determining that the set of event types does not include the second event type; and
facilitating transmission of the second event to the receiver device.

7. The computer-implemented method of claim 1, wherein identifying the event-suppression configuration includes detecting one or more user interactions with a graphical user-interface element displayed on the sender device.

8. A system comprising:
one or more processors; and
memory storing thereon instructions that, as a result of being executed by the one or more processors, cause the system to perform operations comprising:
receiving sensor data collected by one or more sensors associated with a sender device;
determining an occurrence of an event associated with a subject of the sender device, wherein the event is determined based on the sensor data, and wherein the event is associated with a first event type;
identifying an event-suppression configuration associated with a receiver device, wherein the event-suppression configuration identifies a set of event types;
applying the event-suppression configuration to the event associated with the subject, wherein applying the event-suppression configuration includes determining whether the set of event types includes the first event type;
determining that the set of event types includes the first event type; and
suppressing transmission of the event to the receiver device.

9. The system of claim 8, wherein the event is associated with a severity metric, and wherein the instructions further cause the system to perform operations comprising:
identifying a second event-suppression configuration associated with a second receiver device, wherein the second event-suppression configuration identifies a severity threshold;
applying the second event-suppression configuration to the event associated with the subject, wherein applying the second event-suppression configuration includes determining whether the severity metric exceeds the severity threshold;
determining that the severity metric does not exceed the severity threshold; and
suppressing transmission of the event to the second receiver device.

10. The system of claim 8, wherein the sensor data indicates that the sender device was located at a particular location during the occurrence of the event, and wherein the instructions further cause the system to perform operations comprising:
identifying a third event-suppression configuration associated with a third receiver device, wherein the third event-suppression configuration identifies a geographic region;
applying the third event-suppression configuration to the event associated with the subject, wherein applying the third event-suppression configuration includes determining whether the particular location is within the geographic region;
determining that the particular location is within the geographic region; and
suppressing transmission of the event to the third receiver device.

11. The system of claim 8, wherein the event is associated with a frequency metric, and wherein the instructions further cause the system to perform operations comprising:
identifying a fourth event-suppression configuration associated with a fourth receiver device, wherein the fourth event-suppression configuration an event-frequency threshold;
applying the fourth event-suppression configuration to the event associated with the subject, wherein applying the fourth event-suppression configuration includes determining whether the frequency metric exceeds the event-frequency threshold;
determining that the frequency metric does not exceed the event-frequency threshold; and
suppressing transmission of the event to the fourth receiver device.

12. The system of claim 8, wherein determining the occurrence of the event includes applying a machine-learning model to the sensor data to determine the occurrence of the event.

13. The system of claim 8, wherein the instructions further cause the system to perform operations comprising:
determining an occurrence of a second event associated with the subject, wherein the second event is determined based on the sensor data, and wherein the second event is associated with a second event type;
applying the event-suppression configuration to the second event associated with the subject, wherein applying the event-suppression configuration includes determining whether the set of event types includes the second event type;
determining that the set of event types does not include the second event type; and
facilitating transmission of the second event to the receiver device.

14. The system of claim 8, wherein identifying the event-suppression configuration includes detecting one or more user interactions with a graphical user-interface element displayed on the sender device.

15. A non-transitory, computer-readable storage medium storing thereon executable instructions that, as a result of being executed by one or more processors of a computer system, cause the computer system to perform operations comprising:
receiving sensor data collected by one or more sensors associated with a sender device;
determining an occurrence of an event associated with a subject of the sender device, wherein the event is determined based on the sensor data, and wherein the event is associated with a first event type;
identifying an event-suppression configuration associated with a receiver device, wherein the event-suppression configuration identifies a set of event types;
applying the event-suppression configuration to the event associated with the subject, wherein applying the event-suppression configuration includes determining whether the set of event types includes the first event type;
determining that the set of event types includes the first event type; and
suppressing transmission of the event to the receiver device.

16. The non-transitory, computer-readable storage medium of claim 15, wherein the event is associated with a severity metric, and wherein the instructions further cause the computer system to perform operations comprising:
identifying a second event-suppression configuration associated with a second receiver device, wherein the second event-suppression configuration identifies a severity threshold;
applying the second event-suppression configuration to the event associated with the subject, wherein applying the second event-suppression configuration includes determining whether the severity metric exceeds the severity threshold;
determining that the severity metric does not exceed the severity threshold; and
suppressing transmission of the event to the second receiver device.

17. The non-transitory, computer-readable storage medium of claim 15, wherein the sensor data indicates that the sender device was located at a particular location during the occurrence of the event, and wherein the instructions further cause the computer system to perform operations comprising:
identifying a third event-suppression configuration associated with a third receiver device, wherein the third event-suppression configuration identifies a geographic region;
applying the third event-suppression configuration to the event associated with the subject, wherein applying the third event-suppression configuration includes determining whether the particular location is within the geographic region;
determining that the particular location is within the geographic region; and
suppressing transmission of the event to the third receiver device.

18. The non-transitory, computer-readable storage medium of claim 15, wherein the event is associated with a frequency metric, and wherein the instructions further cause the computer system to perform operations comprising:
identifying a fourth event-suppression configuration associated with a fourth receiver device, wherein the fourth event-suppression configuration an event-frequency threshold;
applying the fourth event-suppression configuration to the event associated with the subject, wherein applying the fourth event-suppression configuration includes determining whether the frequency metric exceeds the event-frequency threshold;
determining that the frequency metric does not exceed the event-frequency threshold; and
suppressing transmission of the event to the fourth receiver device.

19. The non-transitory, computer-readable storage medium of claim 15, wherein determining the occurrence of the event includes applying a machine-learning model to the sensor data to determine the occurrence of the event.

20. The non-transitory, computer-readable storage medium of claim 15, wherein the instructions further cause the computer system to perform operations comprising:
determining an occurrence of a second event associated with the subject, wherein the second event is determined based on the sensor data, and wherein the second event is associated with a second event type;
applying the event-suppression configuration to the second event associated with the subject, wherein applying the event-suppression configuration includes determining whether the set of event types includes the second event type;
determining that the set of event types does not include the second event type; and
facilitating transmission of the second event to the receiver device.

21. The non-transitory, computer-readable storage medium of claim 15, wherein identifying the event-suppression configuration includes detecting one or more user interactions with a graphical user-interface element displayed on the sender device.
